SEQUENCE LISTING (62 sequences)

SEQ ID NO: 1 - protein, 6 aa, Homo sapiens
  SITE (1)..(6): CDR1 of Light Chain(1584)
QDIDIY

SEQ ID NO: 2 - protein, 3 aa, Homo sapiens
  SITE (1)..(3): CDR2 of Light Chain(1584)
AAS

SEQ ID NO: 3 - protein, 9 aa, Homo sapiens
  SITE (1)..(9): CDR3 of Light Chain(1584)
QQLTTYPLT

SEQ ID NO: 4 - protein, 8 aa, Homo sapiens
  SITE (1)..(8): CDR1 of Heavy Chain(1584)
GFTFSNYG

SEQ ID NO: 5 - protein, 8 aa, Homo sapiens
  SITE (1)..(8): CDR2 of Heavy Chain(1584)
ISSHGRDK

SEQ ID NO: 6 - protein, 13 aa, Homo sapiens
  SITE (1)..(13): CDR3 of Heavy Chain(1584)
AKDKGVKYSYGDS

SEQ ID NO: 7 - protein, 106 aa, Homo sapiens
  SITE (1)..(106): Variable Region of Light Chain(1584)
DIQLTQSPSFLSASVGDRVTITCRASQDIDIYLAWYQQKPGKAPNLLIYAASTLQSGVPLRFSGSGSGTEFTLTISSLQPEDFATYYCQQLTTYPLTFGGGTTVDI

SEQ ID NO: 8 - protein, 120 aa, Homo sapiens
  SITE (1)..(120): Variable Region of Heavy Chain(1584)
QVQLVESGGGVVEPGRSLRLSCAASGFTFSNYGMHWVRQAPGKGLDWVAVISSHGRDKYYADSVKGRFTISRDYSRNMVYLQMNSLRPEDTAVYYCAKDKGVKYSYGDSWGRGTLVTVSS

SEQ ID NO: 9 - protein, 6 aa, Homo sapiens
  SITE (1)..(6): CDR1 of Light Chain(1573)
QSISTW

SEQ ID NO: 10 - protein, 3 aa, Homo sapiens
  SITE (1)..(3): CDR2 of Light Chain(1573)
KAS

SEQ ID NO: 11 - protein, 9 aa, Homo sapiens
  SITE (1)..(9): CDR3 of Light Chain(1573)
QQYDRYSLT

SEQ ID NO: 12 - protein, 8 aa, Homo sapiens
  SITE (1)..(8): CDR1 of Heavy Chain(1573)
GFTFSSHG

SEQ ID NO: 13 - protein, 8 aa, Homo sapiens
  SITE (1)..(8): CDR2 of Heavy Chain(1573)
VSNDGSDK

SEQ ID NO: 14 - protein, 13 aa, Homo sapiens
  SITE (1)..(13): CDR3 of Heavy Chain(1573)
AKGSIAARQYFDY

SEQ ID NO: 15 - protein, 107 aa, Homo sapiens
  SITE (1)..(107): Variable Region of Light Chain(1573)
DIQMTQSPSTLSASVGDRVTITCRASQSISTWLAWYQQKPGKAPELLIYKASNLESGVPSRFSSSGSGTEFTLTISSLQPDDFATYYCQQYDRYSLTFGGGTKVEIK

SEQ ID NO: 16 - protein, 120 aa, Homo sapiens
  SITE (1)..(120): Variable Region of Heavy Chain(1573)
QVQLVESGGGVVQPGRSLRLSCAASGFTFSSHGMHWVRQAPGKGLEWVAAVSNDGSDKYYADSVKGRLTISRDNSENTLYLQMSSLRGEDTAVYYCAKGSIAARQYFDYWGQGTLVTVSS

SEQ ID NO: 17 - protein, 6 aa, Homo sapiens
  SITE (1)..(6): CDR1 of Light Chain(1572)
QDVGIW

SEQ ID NO: 18 - protein, 3 aa, Homo sapiens
  SITE (1)..(3): CDR2 of Light Chain(1572)
KTS

SEQ ID NO: 19 - protein, 9 aa, Homo sapiens
  SITE (1)..(9): CDR3 of Light Chain(1572)
QQHGAFPIT

SEQ ID NO: 20 - protein, 8 aa, Homo sapiens
  SITE (1)..(8): CDR1 of Heavy Chain(1572)
GFIFNNYA

SEQ ID NO: 21 - protein, 8 aa, Homo sapiens
  SITE (1)..(8): CDR2 of Heavy Chain(1572)
ISFDGRDK

SEQ ID NO: 22 - protein, 13 aa, Homo sapiens
  SITE (1)..(13): CDR3 of Heavy Chain(1572)
ATGSKEAHQFVLY

SEQ ID NO: 23 - protein, 107 aa, Homo sapiens
  SITE (1)..(107): Variable Region of Light Chain(1572)
DIQMTQSPSTLSASVGDRVTITCRASQDVGIWLAWYQQKPGKAPNSLIYKTSSLHNGVPSRFSGSGYGTEFTLTISSLQPDDFATYYCQQHGAFPITFGQGTRLEIR

SEQ ID NO: 24 - protein, 120 aa, Homo sapiens
  SITE (1)..(120): Variable Region of Heavy Chain(1572)
QVQLVESGGGVVQPGRSQRLSCVASGFIFNNYAMHWVRQAPGKGLEWVAAISFDGRDKFYADSVQGRFSISRDNSRDTLYLQMNGLRPDDTAVYFCATGSKEAHQFVLYWAQGTLVTVSS

SEQ ID NO: 25 - protein, 6 aa, Homo sapiens
  SITE (1)..(6): CDR1 of Light Chain(1587)
QSISTW

SEQ ID NO: 26 - protein, 3 aa, Homo sapiens
  SITE (1)..(3): CDR2 of Light Chain(1587)
KAS

SEQ ID NO: 27 - protein, 9 aa, Homo sapiens
  SITE (1)..(9): CDR3 of Light Chain(1587)
QQYDRYSLT

SEQ ID NO: 28 - protein, 8 aa, Homo sapiens
  SITE (1)..(8): CDR1 of Heavy Chain(1587)
GFTFSSHG

SEQ ID NO: 29 - protein, 8 aa, Homo sapiens
  SITE (1)..(8): CDR2 of Heavy Chain(1587)
VSNDGSDK

SEQ ID NO: 30 - protein, 13 aa, Homo sapiens
  SITE (1)..(13): CDR3 of Heavy Chain(1587)
AKGSIAARQYFDC

SEQ ID NO: 31 - protein, 107 aa, Homo sapiens
  SITE (1)..(107): Variable Region of Light Chain(1587)
DIQMTQSPSTLSASVGDRVTITCRASQSISTWLAWYQQKPGKAPELLIYKASNLESGVPSRFSGSGSGTEFTLTISSLQPDDFATYYCQQYDRYSLTFGGGTKVEIK

SEQ ID NO: 32 - protein, 120 aa, Homo sapiens
  SITE (1)..(120): Variable Region of Heavy Chain(1587)
QVQLVESGGGVVRPGRSLRLSCAASGFTFSSHGMHWVRQAPGKGLEWVAAVSNDGSDKYYADSVKGRLTISRDNSENTLYLQMSSLRGEDTAVYYCAKGSIAARQYFDCWGQGTLVTVSS

SEQ ID NO: 33 - DNA, 318 bp, Homo sapiens
  V_region (1)..(318): Variable Region of Light Chain(1584)
gacatccagctgacccagagccccagcttcctgagcgccagcgtgggcgaccgggtgaccatcacctgccgggccagccaggacatcgacatctacctggcctggtatcagcagaagcccggcaaggcccccaacctgctgatctacgccgccagcaccctgcagagcggcgtgcccctgcggtttagcggcagcggctccggcaccgagttcaccctgaccatcagcagcctgcagcccgaggacttcgccacctactactgccagcagctgaccacctaccccctgacctttggcggcggaaccaccgtggacatc

SEQ ID NO: 34 - DNA, 360 bp, Homo sapiens
  V_region (1)..(360): Variable Region of Heavy Chain(1584)
caggtgcagctggtcgagtctggcggcggagtggtggagcccggcagaagcctgagactgagctgcgccgccagcggcttcaccttcagcaactacggcatgcactgggtgcggcaggccccaggcaagggactggactgggtggccgtgatcagcagccacggccgggacaagtactacgccgacagcgtgaagggccggttcaccatcagccgggactacagccggaacatggtgtacctgcagatgaacagcctgcggcccgaggacaccgccgtgtactactgcgccaaggacaagggcgtgaagtacagctacggcgacagctggggcaggggcaccctggtgaccgtctcgagt

SEQ ID NO: 35 - DNA, 321 bp, Homo sapiens
  V_region (1)..(321): Variable Region of Light Chain(1573)
gacatccagatgacccagagccccagcaccctgagcgccagcgtgggcgaccgggtgaccatcacctgcagagcaagccagagcatcagcacatggctcgcttggtatcagcagaagcccggcaaggcccccgagctgctgatctacaaggccagcaacctggaaagcggcgtgcccagccggttcagcagcagcggcagcggcaccgagttcaccctgaccatcagcagcctgcagcccgacgacttcgccacctactactgccagcagtacgaccggtacagcctgacctttggcggcggaacaaaggtggagatcaag

SEQ ID NO: 36 - DNA, 360 bp, Homo sapiens
  V_region (1)..(360): Variable Region of Heavy Chain(1573)
caggtgcagctggtcgagtctggcggcggagtggtgcagcccggcagaagcctgagactgagctgcgccgccagcggcttcaccttcagcagccacggcatgcactgggtgcggcaggcccctggcaagggactggaatgggtggccgccgtgagcaacgacggcagcgacaagtactacgccgacagcgtgaagggccggctgaccatcagccgggacaacagcgagaacaccctgtacctgcagatgagcagcctgcggggcgaggacaccgccgtgtactactgcgccaagggcagcattgccgccaggcagtacttcgactactggggccagggcaccctggtgaccgtctcgagt

SEQ ID NO: 37 - DNA, 321 bp, Homo sapiens
  V_region (1)..(321): Variable Region of Light Chain(1572)
gacatccagatgacccagagccccagcaccctgagcgccagcgtgggcgaccgggtgaccatcacctgccgggccagccaggacgtgggcatctggctggcctggtatcagcagaagcccggcaaggcccccaacagcctgatctacaagaccagcagcctgcacaacggcgtgcccagccggtttagcggcagcggctacggcaccgagttcaccctgaccatcagctccctgcagcccgacgacttcgccacctactactgccagcagcacggcgccttccccatcaccttcggccagggcacccggctggaaatccgg

SEQ ID NO: 38 - DNA, 360 bp, Homo sapiens
  V_region (1)..(360): Variable Region of Heavy Chain(1572)
caggtgcagctggtcgagtctggcggcggagtggtgcagcccggcagaagccagagactgagctgcgtggccagcggcttcatcttcaacaactacgccatgcactgggtgcggcaggctccaggcaagggactggaatgggtggccgccatcagcttcgacggccgggacaagttctacgccgacagcgtgcagggccggttcagcatcagccgggacaacagcagggacaccctgtacctgcagatgaacggcctgcggcccgacgacaccgccgtgtacttttgcgccaccggcagcaaagaggcccaccagttcgtcctgtactgggcccagggcaccctggtgaccgtctcgagt

SEQ ID NO: 39 - DNA, 321 bp, Homo sapiens
  V_region (1)..(321): Variable Region of Light Chain(1587)
gacatccagatgacccagagccccagcaccctgagcgccagcgtgggcgaccgggtgaccatcacctgccgggcctcccagagcatctccacctggctggcctggtatcagcagaagcccggcaaggcccccgagctgctgatctacaaggccagcaacctggaaagcggcgtgcccagccggtttagcggcagcggctccggcaccgagttcaccctgaccatcagcagcctgcagcccgacgacttcgccacctactactgccagcagtacgaccggtacagcctgacctttggcggcggaacaaaggtggagatcaag

SEQ ID NO: 40 - DNA, 360 bp, Homo sapiens
  V_region (1)..(360): Variable Region of Heavy Chain(1587)
caggtgcagctggtcgagtctggcggcggagtggtgcggcctggcagaagcctgagactgagctgcgccgccagcggcttcaccttcagcagccacggcatgcactgggtgcggcaggccccaggcaaaggcctggaatgggtggccgccgtgagcaacgacggcagcgacaagtactacgccgacagcgtgaagggccggctgaccatcagccgggacaacagcgagaacaccctgtacctgcagatgagcagcctgcggggcgaggacaccgccgtgtactactgcgccaagggcagcattgccgccaggcagtacttcgactgctggggccagggcaccctggtgaccgtctcgagt

SEQ ID NO: 41 - DNA, 38 bp, Artificial Sequence
  Synthetic polynucleotide
atatatatgcggccgcttattaacactctcccctgttg

SEQ ID NO: 42 - DNA, 20 bp, Artificial Sequence
  Synthetic polynucleotide
gacsgatgggcccttggtgg

SEQ ID NO: 43 - DNA, 20 bp, Artificial Sequence
  Synthetic polynucleotide
gagtggctcctgggggaaga

SEQ ID NO: 44 - DNA, 36 bp, Artificial Sequence
  Synthetic polynucleotide
tattcccatggcgcgcccagrtgcagctggtgcart

SEQ ID NO: 45 - DNA, 36 bp, Artificial Sequence
  Synthetic polynucleotide
tattcccatggcgcgccsaggtccagctggtrcagt

SEQ ID NO: 46 - DNA, 36 bp, Artificial Sequence
  Synthetic polynucleotide
tattcccatggcgcgcccagrtcaccttgaaggagt

SEQ ID NO: 47 - DNA, 35 bp, Artificial Sequence
  Synthetic polynucleotide
tattcccatggcgcgccsaggtgcagctggtggag

SEQ ID NO: 48 - DNA, 36 bp, Artificial Sequence
  Synthetic polynucleotide
tattcccatggcgcgcccaggtgcagctacagcagt

SEQ ID NO: 49 - DNA, 36 bp, Artificial Sequence
  Synthetic polynucleotide
tattcccatggcgcgcccagstgcagctgcaggagt

SEQ ID NO: 50 - DNA, 36 bp, Artificial Sequence
  Synthetic polynucleotide
tattcccatggcgcgccgargtgcagctggtgcagt

SEQ ID NO: 51 - DNA, 37 bp, Artificial Sequence
  Synthetic polynucleotide
tattcccatggcgcgcccaggtacagctgcagcagtc

SEQ ID NO: 52 - DNA, 45 bp, Artificial Sequence
  Synthetic polynucleotide
ggcgcgccatgggaatagctagccgacatccagwtgacccagtct

SEQ ID NO: 53 - DNA, 45 bp, Artificial Sequence
  Synthetic polynucleotide
ggcgcgccatgggaatagctagccgatgttgtgatgactcagtct

SEQ ID NO: 54 - DNA, 45 bp, Artificial Sequence
  Synthetic polynucleotide
ggcgcgccatgggaatagctagccgaaattgtgwtgacrcagtct

SEQ ID NO: 55 - DNA, 45 bp, Artificial Sequence
  Synthetic polynucleotide
ggcgcgccatgggaatagctagccgatattgtgatgacccacact

SEQ ID NO: 56 - DNA, 43 bp, Artificial Sequence
  Synthetic polynucleotide
ggcgcgccatgggaatagctagccgaaacgacactcacgcagt

SEQ ID NO: 57 - DNA, 45 bp, Artificial Sequence
  Synthetic polynucleotide
ggcgcgccatgggaatagctagccgaaattgtgctgactcagtct

SEQ ID NO: 58 - DNA, 51 bp, Artificial Sequence
  Synthetic polynucleotide
accgcctccaccggcggccgcttattaacactctcccctgttgaagctctt

SEQ ID NO: 59 - DNA, 30 bp, Artificial Sequence
  Synthetic polynucleotide
ggaggcgctcgagacggtgaccagggtgcc

SEQ ID NO: 60 - DNA, 30 bp, Artificial Sequence
  Synthetic polynucleotide
ggaggcgctcgagacggtgaccattgtccc

SEQ ID NO: 61 - DNA, 30 bp, Artificial Sequence
  Synthetic polynucleotide
ggaggcgctcgagacggtgaccagggttcc

SEQ ID NO: 62 - DNA, 30 bp, Artificial Sequence
  Synthetic polynucleotide
ggaggcgctcgagacggtgaccgtggtccc

(19) United States
(12) Patent Application Publication
(10) Pub. No.: US 2013/0045207 A1
(43) Pub. Date: Feb. 21, 2013

(54) ANTIBODY AGAINST SEROTYPE G LIPOPOLYSACCHARIDE OF PSEUDOMONAS AERUGINOSA

(71) Applicants: Jiro Tanaka, Yokohama-shi (JP); Peter Sejer Andersen, Vanlose (DK); Takafumi Okutomi, Yokohama-shi (JP); Tsuneyoshi Inaba, Yokohama-shi (JP); Keiko Otsuka, Yokohama-shi (JP); Hirotomo Akabane, Yokohama-shi (JP); Yukari Hoshina, Yokohama-shi (JP); Hiroshi Nagaso, Yokohama-shi (JP); Masashi Kumagai, Yokohama-shi (JP)

(72) Inventors: Jiro Tanaka, Yokohama-shi (JP); Peter Sejer Andersen, Vanlose (DK); Takafumi Okutomi, Yokohama-shi (JP); Tsuneyoshi Inaba, Yokohama-shi (JP); Keiko Otsuka, Yokohama-shi (JP); Hirotomo Akabane, Yokohama-shi (JP); Yukari Hoshina, Yokohama-shi (JP); Hiroshi Nagaso, Yokohama-shi (JP); Masashi Kumagai, Yokohama-shi (JP)

(73) Assignees: SYMPHOGEN A/S, Lyngby (DK); MEIJI SEIKA PHARMA CO., LTD., Chuo-ku, Tokyo (JP)

(21) Appl. No.: 13/579,748

(22) PCT Filed: Feb. 18, 2011
(86) PCT No.: PCT/JP2011/054229

§ 371 Date: Sep. 26, 2012

(30) Foreign Application Priority Data
Feb. 18, 2010 (JP) 2010-033429

(51) Int. Cl. C07K 16/12; A61P 31/04; A61K 39/40; C12N 5/12; C12N 15/13

(52) U.S. Cl. 424/137.1; 435/329; 530/387.5; 536/23.53

(57) ABSTRACT

Provided is a novel antibody having an excellent antibacterial activity against P. aeruginosa. By using plasmablasts obtained from cystic fibrosis patients with chronic P. aeruginosa pulmonary infection as starting materials, antibodies which bind to LPS of a P. aeruginosa strain of serotype G and which have excellent antibacterial activities in vitro and in vivo were successfully obtained.

### TECHNICAL FIELD

The present invention relates to an antibody against serotype G lipopolysaccharide of P. aeruginosa and applications thereof. More specifically, the present invention relates to an antibody which specifically binds to serotype G lipopolysaccharide of a P. aeruginosa strain, and a pharmaceutical composition, a diagnostic agent for a P. aeruginosa infection, and a P. aeruginosa detection kit, each including any of the antibodies.

### BACKGROUND ART

P. aeruginosa (Pseudomonas aeruginosa) is a gram-negative aerobic bacillus widely and generally distributed in natural environments such as soil and water. P. aeruginosa is an avirulent bacterium which normally is not pathogenic to healthy subjects, who have a moderate antibody titer and a sufficient immune function against P. aeruginosa. However, once debilitated patients are infected with P. aeruginosa, P. aeruginosa may cause severe symptoms, which may lead to the death of the patients. For this reason, P. aeruginosa has attracted attention as a major causative bacterium of nosocomial infections and opportunistic infections, and hence the prevention and treatment of P. aeruginosa infections have been important issues in the medical field.

For the prevention or treatment of P. aeruginosa infections, antibiotics or synthetic antibacterial agents have mainly been used. However, P. aeruginosa develops resistance to such medicines, and hence such medicines do not provide a sufficient therapeutic effect in many cases. Particularly, treatment of infections with multi-drug resistant P. aeruginosa (MDRP) using antibiotics or the like is difficult, and has limitation. For this reason, as an alternative method thereto, treatment using an immunoglobulin preparation has been conducted.

Meanwhile, the prevention or treatment of a P. aeruginosa infection using an antibody against P. aeruginosa has been examined. For example, antibodies each of which specifically binds to a P. aeruginosa strain of a specific serotype have been developed (Patent Literatures 1 to 5, and Non-Patent Literatures 1 and 2).

However, the antibodies against P. aeruginosa developed so far do not provide a sufficient effect in prevention or treatment of a P. aeruginosa infection.

### CITATION LIST

### Patent Literature

[PTL 1] Japanese Unexamined Patent Application Publication No. Hei 6-178688
[PTL 2] Japanese Unexamined Patent Application Publication No. Hei 6-178689
[PTL 3] Japanese Unexamined Patent Application Publication No. Hei 7-327677
[PTL 4] International Publication No. WO2004/101622
[PTL 5] International Publication No. WO2006/084758

### Non Patent Literature

[NPL 1] The Journal of Infectious Diseases, 152, 6, 1985, 1290-1299.
[NPL 2] Journal of General Microbiology, 133, 1987, 3581-3590.

### SUMMARY OF INVENTION

### Technical Problem

The present invention has been made in view of the above-described circumstances, and an object of the present invention is to provide a novel antibody which has an excellent antibacterial activity against P. aeruginosa. One main object of the present invention is to provide an antibody which specifically binds to serotype G lipopolysaccharide of a P. aeruginosa strain

### Solution to Problem

To achieve the above-described object, the present inventors employed the following approach. First, blood samples were collected from cystic fibrosis patients with chronic P. aeruginosa pulmonary infection and healthy volunteers. Donor specimens having a high proportion of plasmablasts which were specific to lipopolysaccharide (hereinafter sometimes simply referred to as “LPS”) were identified by: (1) FACS analysis which determined the amounts of plasmablasts and plasmacytes in the circulating blood; (2) ELISPOT analysis which determined the amount of cells, in the circulating blood, producing antibodies specific to a specific LPS antigen; and (3) ELISA analysis which determined the presence or absence of immunoglobulins specific to a specific LPS antigen. Next, antibodies which recognized LPS were prepared from the donor specimens thus identified.

Specifically, viable plasmablasts were selected by staining CD19, CD38, λ light chain, and dead cells. On the selected plasmablasts, the pairing of DNA sequences coding a heavy chain variable region (VH) and a light chain variable region (VL) which were originated from the same B cell by two-stage PCR involving multiplex overlap-extension RT-PCR and subsequent nested PCR (FIG. 1). Next, amplified DNA was inserted into a screening vector, and then transformed into Escherichia coli. A repertoire of the amplified vector was purified from the Escherichia coli. The obtained antibody library was expressed in animal culture cells. Clones coding antibodies which bound to purified LPS molecules were screened by ELISA, and LPS-specific clones were selected. Then, the base sequences of the selected clones were determined. Thereafter, antibodies coded by the thus obtained clones were examined for their various activities, their serotype specificity, and epitopes.

As a result, it is found out that identified antibodies bind to serotype G LPS of P. aeruginosa, and have excellent antibacterial activities in vitro and in vivo.

Specifically, the present invention relates to antibodies which bind to serotype G LPS of P. aeruginosa, show an excellent antibacterial activity. The present invention also relates to applications of the antibodies. More specifically, the present invention provides

[1] An antibody which recognizes B-band LPS of lipopolysaccharides of P. aeruginosa, and which substantially binds to a surface of a P. aeruginosa strain of serotype G, but does not substantially binds to any one of surfaces of P. aeruginosa strains of serotype A, B, C, D, E, F, H, I, and M.
[2] The antibody according to clause 1, which has an opsonic activity against a P. aeruginosa strain of serotype G.
[3] The antibody according to clause 2, wherein an EC50 of an opsonic activity against a P. aeruginosa strain identified by ATCC 33354 is 0.5 μg/ml or less.
[4] The antibody according to clause 2, wherein an EC50 of an opsonic activity against a P. aeruginosa strain identified by ATCC 27584 is 3 μg/ml or less.
[5] The antibody according to any one of clauses 1 to 4, which has an agglutination activity against a P. aeruginosa strain of serotype G.
[6] The antibody according to clause 5, wherein an agglutination titer per amount (μg) of IgG against a P. aeruginosa strain identified by ATCC 27584 is 100 or more.
[7] The antibody according to any one of clauses 1 to 6, which has an antibacterial effect against a systemic infection with a P. aeruginosa strain of serotype G.
[8] The antibody according to clause 7, wherein an ED50 of an antibacterial effect on a neutropenic mouse model of systemic infection with a P. aeruginosa strain identified by ATCC 27584 is not more than 1/100 of that of Venilon.
[9] The antibody which has any one of the following features (a) to (d):

(a) comprising
a light chain variable region including amino acid sequences described in SEQ ID NOs: 1 to 3 or the amino acid sequences described in SEQ ID NOs: 1 to 3 in at least one of which one or more amino acids are substituted, deleted, added, and/or inserted, and
a heavy chain variable region including amino acid sequences described in SEQ ID NOs: 4 to 6 or the amino acid sequences described in SEQ ID NOs: 4 to 6 in at least one of which one or more amino acids are substituted, deleted, added, and/or inserted;

(b) comprising
a light chain variable region including amino acid sequences described in SEQ ID NOs: 9 to 11 or the amino acid sequences described in SEQ ID NOs: 9 to 11 in at least one of which one or more amino acids are substituted, deleted, added, and/or inserted, and
a heavy chain variable region including amino acid sequences described in SEQ ID NOs: 12 to 14 or the amino acid sequences described in SEQ ID NOs: 12 to 14 in at least one of which one or more amino acids are substituted, deleted, added, and/or inserted;

(c) comprising
a light chain variable region including amino acid sequences described in SEQ ID NOs: 17 to 19 or the amino acid sequences described in SEQ ID NOs: 17 to 19 in at least one of which one or more amino acids are substituted, deleted, added, and/or inserted, and
a heavy chain variable region including amino acid sequences described in SEQ ID NOs: 20 to 22 or the amino acid sequences described in SEQ ID NOs: 20 to 22 in at least one of which one or more amino acids are substituted, deleted, added, and/or inserted; and

(d) comprising
a light chain variable region including amino acid sequences described in SEQ ID NOs: 25 to 27 or the amino acid sequences described in SEQ ID NOs: 25 to 27 in at least one of which one or more amino acids are substituted, deleted, added, and/or inserted, and
a heavy chain variable region including amino acid sequences described in SEQ ID NOs: 28 to 30 or the amino acid sequences described in SEQ ID NOs: 28 to 30 in at least one of which one or more amino acids are substituted, deleted, added, and/or inserted.
   [10] The antibody which has any one of the following features (a) to (d):

(a) comprising
a light chain variable region including an amino acid sequence described in SEQ ID NO: 7 or the amino acid sequence described in SEQ ID NO: 7 in which one or more amino acids are substituted, deleted, added, and/or inserted, and
a heavy chain variable region including an amino acid sequence described in SEQ ID NO: 8 or the amino acid sequences described in SEQ ID NO: 8 in which one or more amino acids are substituted, deleted, added, and/or inserted;

(b) comprising
a light chain variable region including an amino acid sequence described in SEQ ID NO: 15 or the amino acid sequences described in SEQ ID NO: 15 in which one or more amino acids are substituted, deleted, added, and/or inserted, and
a heavy chain variable region including an amino acid sequence described in SEQ ID NO: 16 or the amino acid sequences described in SEQ ID NO: 16 in which one or more amino acids are substituted, deleted, added, and/or inserted;

(c) comprising
a light chain variable region including an amino acid sequence described in SEQ ID NO: 23 or the amino acid sequences described in SEQ ID NO: 23 in which one or more amino acids are substituted, deleted, added, and/or inserted, and
a heavy chain variable region including an amino acid sequence described in SEQ ID NO: 24 or the amino acid sequences described in SEQ ID NO: 24 in which one or more amino acids are substituted, deleted, added, and/or inserted; and

(d) comprising
a light chain variable region including an amino acid sequence described in SEQ ID NO: 31 or the amino acid sequences described in SEQ ID NO: 31 in which one or more amino acids are substituted, deleted, added, and/or inserted, and
a heavy chain variable region including an amino acid sequence described in SEQ ID NO: 32 or the amino acid sequence described in SEQ ID NO: 32 in which one or more amino acids are substituted, deleted, added, and/or inserted.
   [11] A peptide comprising a light chain or a light chain variable region of the antibody which has anyone of the following features (a) to (d):

(a) comprising amino acid sequences described in SEQ ID NOs: 1 to 3 or the amino acid sequences described in SEQ ID NOs: 1 to 3 in at least one of which one or more amino acids are substituted, deleted, added, and/or inserted;

(b) comprising amino acid sequences described in SEQ ID NOs: 9 to 11 or the amino acid sequences described in SEQ ID NOs: 9 to 11 in at least one of which one or more amino acids are substituted, deleted, added, and/or inserted;

(c) comprising amino acid sequences described in SEQ ID NOs: 17 to 19 or the amino acid sequences described in SEQ ID NOs: 17 to 19 in at least one of which one or more amino acids are substituted, deleted, added, and/or inserted; and

(d) comprising amino acid sequences described in SEQ ID NOs: 25 to 27 or the amino acid sequences described in SEQ ID NOs: 25 to 27 in at least one of which one or more amino acids are substituted, deleted, added, and/or inserted.

[12] A peptide comprising a light chain or a light chain variable region of the antibody, the peptide having any one of the following features (a) to (d):

(a) comprising an amino acid sequence described in SEQ ID NO: 7 or the amino acid sequence described in SEQ ID NO: 7 in which one or more amino acids are substituted, deleted, added, and/or inserted;

(b) comprising an amino acid sequence described in SEQ ID NO: 15 or the amino acid sequences described in SEQ ID NO: 15 in which one or more amino acids are substituted, deleted, added, and/or inserted;

(c) comprising an amino acid sequence described in SEQ ID NO: 23 or the amino acid sequences described in SEQ ID NO: 23 in which one or more amino acids are substituted, deleted, added, and/or inserted; and

(d) comprising an amino acid sequence described in SEQ ID NO: 31 or the amino acid sequences described in SEQ ID NO: 31 in which one or more amino acids are substituted, deleted, added, and/or inserted.

[13] A peptide comprising a heavy chain or a heavy chain variable region of the antibody, which has any one of the following features (a) to (d):

(a) comprising amino acid sequences described in SEQ ID NOs: 4 to 6 or the amino acid sequences described in SEQ ID NOs: 4 to 6 in at least one of which one or more amino acids are substituted, deleted, added, and/or inserted;

(b) comprising amino acid sequences described in SEQ ID NOs: 12 to 14 or the amino acid sequences described in SEQ ID NOs: 12 to 14 in at least one of which one or more amino acids are substituted, deleted, added, and/or inserted;

(c) comprising amino acid sequences described in SEQ ID NOs: 20 to 22 or the amino acid sequences described in SEQ ID NOs: 20 to 22 in at least one of which one or more amino acids are substituted, deleted, added, and/or inserted; and

(d) comprising amino acid sequences described in SEQ ID NOs: 28 to 30 or the amino acid sequences described in SEQ ID NOs: 28 to 30 in at least one of which one or more amino acids are substituted, deleted, added, and/or inserted.

[14] A peptide comprising a heavy chain or a heavy chain variable region of the antibody, which has any one of the following features (a) to (d):

(a) comprising an amino acid sequence described in SEQ ID NO: 8 or the amino acid sequences described in SEQ ID NO: 8 in which one or more amino acids are substituted, deleted, added, and/or inserted;

(b) comprising an amino acid sequence described in SEQ ID NO: 16 or the amino acid sequences described in SEQ ID NO: 16 in which one or more amino acids are substituted, deleted, added, and/or inserted;

(c) comprising an amino acid sequence described in SEQ ID NO: 24 or the amino acid sequences described in SEQ ID NO: 24 in which one or more amino acids are substituted, deleted, added, and/or inserted; and

(d) comprising an amino acid sequence described in SEQ ID NO: 32 or the amino acid sequence described in SEQ ID NO: 32 in which one or more amino acids are substituted, deleted, added, and/or inserted.

[15] An antibody which binds to an epitope, in B-band LPS of lipopolysaccharides of a P. aeruginosa strain of serotype G, of an antibody described in any one of the following (a) to (d):

(a) an antibody comprising a light chain variable region including an amino acid sequence described in SEQ ID NO: 7 and a heavy chain variable region including an amino acid sequence described in SEQ ID NO: 8;

(b) an antibody comprising a light chain variable region including an amino acid sequence described in SEQ ID NO: 15 and a heavy chain variable region including an amino acid sequence described in SEQ ID NO: 16;

(c) an antibody comprising a light chain variable region including an amino acid sequence described in SEQ ID NO: 23 and a heavy chain variable region including an amino acid sequence described in SEQ ID NO: 24; and

(d) an antibody comprising a light chain variable region including an amino acid sequence described in SEQ ID NO: 31 and a heavy chain variable region including an amino acid sequence described in SEQ ID NO: 32.

[16] A DNA which codes the antibody or the peptide according to any one of clauses 1 to 15.
[17] A hybridoma which produces the antibody according to any one of clauses 1 to 10, and 15.
[18] A pharmaceutical composition for a disease associated with P. aeruginosa, the pharmaceutical composition comprising:

the antibody according any one of clauses 1 to 10, and 15; and optionally

at least one pharmaceutically acceptable carrier and/or diluent.

[19] The pharmaceutical composition according to clause 18, wherein the disease associated with P. aeruginosa is a systemic infectious disease caused by a P. aeruginosa infection.
[20] The pharmaceutical composition according to clause 18, wherein the disease associated with P. aeruginosa is a pulmonary infectious disease caused by a P. aeruginosa infection.
[21] A diagnostic agent for detection of P. aeruginosa, the diagnostic agent comprising: the antibody according any one of clauses 1, 9, 10, and 15.
[22] A kit for detection of P. aeruginosa, the kit comprising: the antibody according any one of clauses 1, 9, 10, and 15.

### ADVANTAGEOUS EFFECTS OF INVENTION

The present invention provides an antibody which binds to serotype G LPS of P. aeruginosa, and which exhibits an excellent antibacterial activity. The antibody of the present invention can exhibit an excellent opsonic effect and an excellent antibacterial effect against a systemic infection or pulmonary infection with P. aeruginosa. The antibody of the present invention can be prepared as a human antibody, and hence is highly safe. Meanwhile, it is possible to prepare a revolutionary polyclonal antibody preparation which is effective for 70% or more of clinically isolated strains, and which exhibits a potent antibacterial activity by combining antibodies of the present invention. The use of an antibody of the present invention makes it possible to effectively treat or prevent infections, such as HAP/VAP, bacteremia, septicemia, and burn wound infection, which are caused by P. aeruginosa, including multi-drug resistant P. aeruginosa.

### BRIEF DESCRIPTION OF DRAWINGS

FIG. 1 is a diagram showing two-stage PCR performed to obtain DNA coding an antibody of the present invention.

FIG. 2 is a diagram showing an OO-VP-002 vector used for the pairing of sequences coding a heavy chain variable region (VH) and a light chain variable region (VL), which were originated from the same B cell.

### DESCRIPTION OF EMBODIMENTS

The present invention provides a novel antibody which binds to serotype G LPS of P. aeruginosa. An “antibody” in the present invention includes all classes and all subclasses of immunoglobulins. The “antibody” includes a polyclonal antibody and a monoclonal antibody, and also includes the form of a functional fragment of an antibody. A “polyclonal antibody” refers to an antibody preparation comprising different kinds of antibodies against different epitopes. Meanwhile, a “monoclonal antibody” means an antibody (including antibody fragments) obtained from a substantially homogeneous population of antibodies. In contrast to the polyclonal antibody, the monoclonal antibody recognizes a single determinant on an antigen. The polyclonal antibody in the present invention also includes a combination of multiple monoclonal antibodies capable of recognizing multiple epitopes on an antigen. The antibody of the present invention is an isolated antibody, that is, an antibody which is separated and/or recovered from components in a natural environment.

A “lipopolysaccharide (LPS)” to which the antibody of the present invention binds is a constituent of an outer membrane of a cell wall of a Gram-negative bacterium, and is a substance formed of a lipid and a polysaccharide (a glycolipid). The carbohydrate chain is formed of a moiety called a core polysaccharide (or a core oligosaccharide), and a moiety called an O antigen (an O side chain polysaccharide). “A-band LPS” is a LPS whose polysaccharide forming the O antigen has the following structure. Specifically, in the structure, units each consisting of “3)-α-D-Rha-(1→2)-α-D-Rha-(1→3)-α-D-Rha-(1” are repeated. In these units, the D-rhamnose is linked by α-1,2 and α-1,3 bonds. The structural formula thereof is shown below; however, the branching mode of D-rhamnose linked by α-1,2-bonds and D-rhamnose linked by α-1,3-bonds is not limited to that shown below.

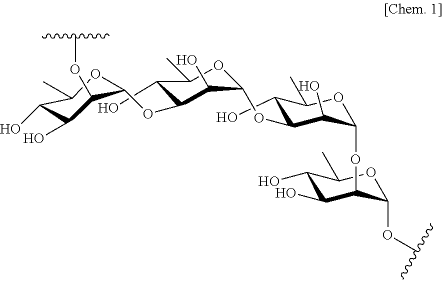

Meanwhile, “B-band LPS” is serotype-specific LPS having a structure in which units each consisting of bonds of two to five sugars in polysaccharide forming the 0 antigen are repeated. As will be described below, the structure of the repeating units in the B-band LPS of P. aeruginosa strains are different from one another, depending on their serotypes (refer to Microbial. Mol. Biol. Rev. 63 523-553 (1999)).

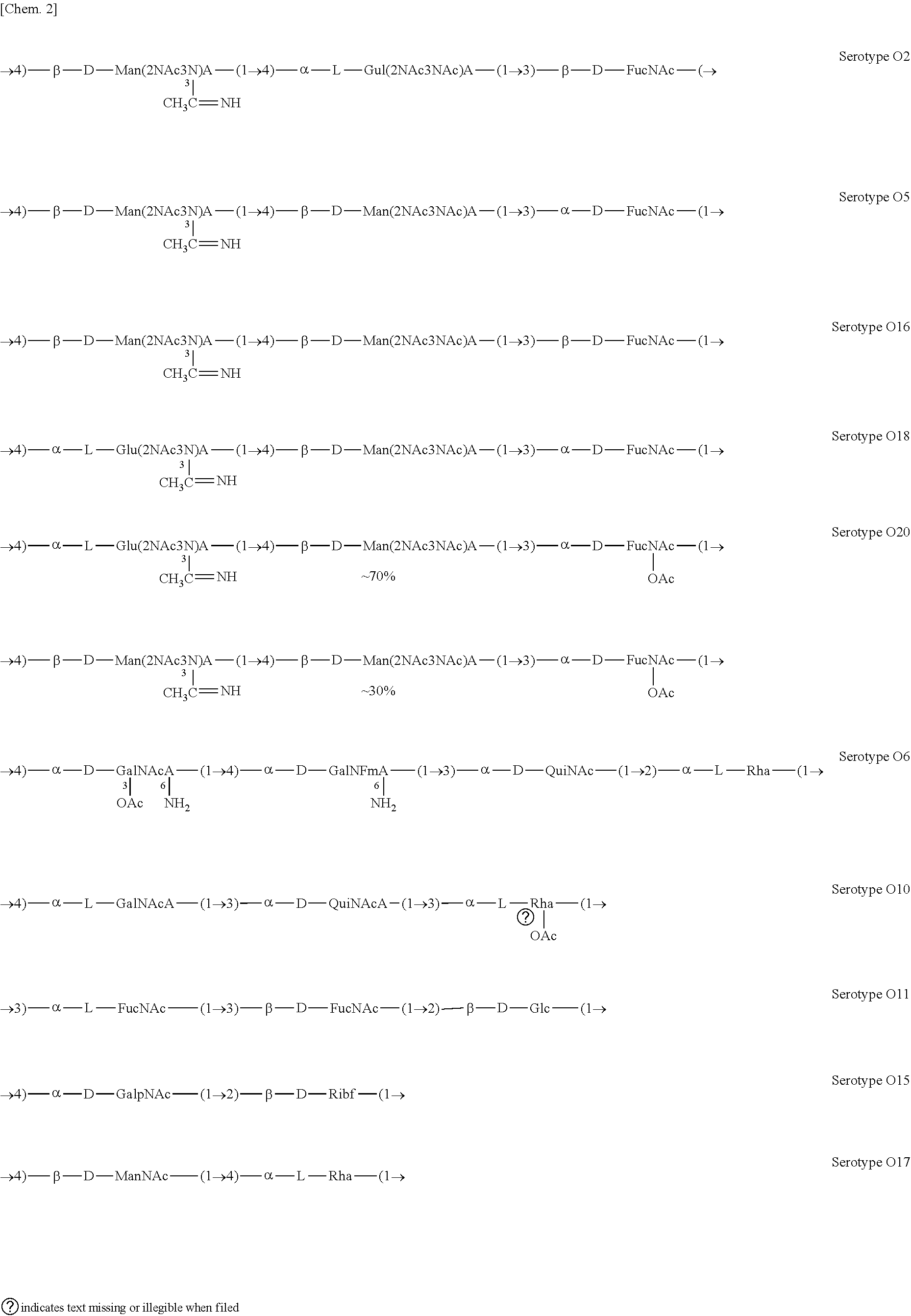

A “serotype” in the present invention means any known serotype of P. aeruginosa. Table 1 shows the correspondence of groups according to the serotyping committee sponsored by Japan P. aeruginosa Society, with types according to IATS (International Antigenic Typing System), both being currently used for P. aeruginosa strains of different serotypes. The serotype of a P. aeruginosa strain can be determined by using a commercially-available immune serum for grouping of P. aeruginosa.

TABLE 1

| JPAS | IATS |
|---|---|
| I | O1 |
| B | O2 |
| A | O3 |
| F | O4 |
| B | O5 |
| C | O7 |
| G | O6 |
| C | O8 |
| D | O9 |
| H | O10 |
| E | O11 |
| L | O12 |
| K | O13 |
| K | O14 |
| J | O15 |
| B | O16 |
| N | O17 |
| — | O18 |
| — | O19 |
| B | O20 |

JPAS: Japan P. aeruginosa society
IATS: International Antigenic Typing System
Reference Document: Microbiology 17 273-304 (1990)

Out of the antibodies identified in the present invention, an antibody “1584”, an antibody “1573,” an antibody “1572” and an antibody “1587” exhibited an excellent specificity to a P. aeruginosa strain of serotype G. Accordingly, another embodiment of the antibody of the present invention is an antibody which specifically binds to lipopolysaccharide of a P. aeruginosa strain of serotype G (hereinafter referred to as an “anti-serotype G LPS antibody”). The anti-serotype G LPS antibody of the present invention is preferably an antibody which recognizes lipopolysaccharide of P. aeruginosa, and which substantially binds to a surface of a P. aeruginosa strain of serotype G, but does not substantially bind to anyone of surfaces of P. aeruginosa strains of serotype A, B, C, D, E, F, H, I, and M. For the anti-serotype G LPS antibody of the present invention, the phrase “substantially binds to” means, for example, that an absorbance, which is indicative of binding capability, is 0.25 or more, when detected by the whole-cell ELISA method described in the examples of the present application. Meanwhile, the phrase “does not substantially bind to” means, for example, that an absorbance, which is indicative of binding capability, is less than 0.25, when detected by the whole-cell ELISA method described in the examples of the present application.

Examples of P. aeruginosa strains of serotype A include those with ATCC accession Nos. 27577 and 33350. Examples of P. aeruginosa strains of serotype B include those with 27578, 33349, BAA-47, 33352, 33363 and 43732. Examples of P. aeruginosa strains of serotype C include those with 33353, 27317 and 33355. Examples of P. aeruginosa strains of serotype D include those with 27580 and 33356. Examples of P. aeruginosa strains of serotype E include those with 29260 and 33358. Examples of P. aeruginosa strains of serotype F include those with 27582 and 33351. Examples of P. aeruginosa strains of serotype G include those with 27584 and 33354. Examples of P. aeruginosa strains of serotype H include those with 27316 and 33357. Examples of P. aeruginosa strains of serotype I include those with 27586 and 33348. An example of P. aeruginosa strains of serotype J is one with 33362. Examples of P. aeruginosa strains of serotype K include those with 33360 and 33361. An example of P. aeruginosa strains of serotype L is one with 33359. An example of P. aeruginosa strains of serotype M is one with 21636. An example of P. aeruginosa strains of serotype N is one with 33364. Examples of P. aeruginosa strains of the other serotype (O18 type and O19 type) include those with 43390 and 43731.

The anti-serotype G LPS antibody of the present invention is preferably an antibody which substantially binds to only P. aeruginosa of serotype G, but which does not substantially binds to any one of P. aeruginosa strains of the other serotypes, out of the P. aeruginosa strains identified by the ATCC accession numbers shown as examples above. More preferably, the anti-serotype G LPS antibody of the present invention is preferably an antibody which substantially binds to all the P. aeruginosa strains of serotype G, but which does not substantially binds to any one of P. aeruginosa strains of the other serotypes, out of the P. aeruginosa strains identified by the ATCC accession numbers shown as examples above.

According to a preferred embodiment, the anti-serotype G LPS antibody of the present invention has an opsonic activity against P. aeruginosa. The anti-serotype G LPS antibody of the present invention can have an opsonic activity against a P. aeruginosa strain of serotype G, as a reflection of the binding activity to a P. aeruginosa strain of serotype G. In particular, the antibody “1584,” the antibody “1573,” the antibody “1572” and the antibody “1587” of the present invention each exhibited a high opsonic activity against a P. aeruginosa strain of serotype G. Particularly notably, when the opsonic activity of the antibody “1584” of the present invention was evaluated by using the P. aeruginosa strain of serotype G (ATCC 33354) and by employing the detection method using, as an index, a fluorescence intensity of human polymorphonuclear leukocytes incorporating FITC-labeled P. aeruginosa, as described in the examples of the present application, the EC of the antibody “1584” was 0.1 μg/ml. Moreover, when the opsonic activities of the antibody “1573,” the antibody “1572” and the antibody “1587” of the present invention were evaluated by using the P. aeruginosa strain of serotype G (ATCC 27584) and by employing the detection method using, as an index, a fluorescence intensity of human polymorphonuclear leukocytes incorporating FITC-labeled P. aeruginosa, as described in the examples of the present application, the EC50s of the antibody “1573,” the antibody “1572” and the antibody “1587” were 0.72, 1.30 and 2.59 μg/ml, respectively. The anti-serotype G LPS antibody of the present invention preferably has such an excellent opsonic activity, and is, for example, an antibody of which EC50 of opsonic activity against the P. aeruginosa strain of serotype G (ATCC 33354) is 0.5 μg/ml or less (for example, 0.4 μg/ml or less, 0.3 μg/ml or less, 0.2 μg/ml or less or 0.1 μg/ml or less) or an antibody of which EC50 of opsonic activity against the P. aeruginosa strain of serotype G (ATCC 27584) is 3 μg/ml or less (for example, 2 μg/ml or less, 1.5 μg/ml or less, 1 μg/ml or less or 0.8 μg/ml or less).

Moreover, when the opsonic activity of the anti-serotype

G LPS antibody of the present invention is evaluated by using the P. aeruginosa strain of serotype G(ATCC 33354) and by employing the detection method using, as an index, a fluorescence intensity of human polymorphonuclear leukocytes incorporating FITC-labeled P. aeruginosa, as described in the examples of the present application, the mean fluorescence intensity (MFI) value of the anti-serotype G LPS antibody at 30 μg/ml is preferably not less than 0.5 times (for example, not less than 0.7 times, not less than 1 time or not less than 1.3 times) the mean fluorescence intensity (MFI) value of Venilon at 1000 μg/ml. Meanwhile, when the opsonic activity is evaluated by using the P. aeruginosa strain of serotype G (ATCC 27584), the mean fluorescence intensity (MFI) value of the anti-serotype G LPS antibody at 30 μg/ml is preferably not less than (for example, not less than 2 times, not less than 3 times, or not less than 4 times) the mean fluorescence intensity (MFI) value of Venilon at 1000 μg/ml.

According to another preferred embodiment, the anti-serotype G LPS antibody of the present invention has an agglutination activity against P. aeruginosa. The antibody “1584” of the present invention showed an excellent agglutination titer per amount (μg) of IgG of 166, when the P. aeruginosa strain of serotype G (ATCC 27584) was used. Because of having such an excellent agglutination activity, the anti-serotype G LPS antibody of the present invention used as a medicine can induce an efficient opsonic activity even in a low dose, and hence an effect of infection prevention can be anticipated. The anti-serotype G LPS antibody of the present invention preferably has an agglutination titer per amount (μg) of IgG of 100 or more (for example, 110 or more, 130 or more, 150 or more or 160 or more), when the P. aeruginosa strain of serotype G (ATCC 27584) is used.

According to another preferred embodiment, the anti-serotype G LPS antibody of the present invention has an antibacterial effect against a systemic infection with P. aeruginosa. Each of the antibody “1584,” the antibody “1573” and the antibody “1572” of the present invention exhibited an antibacterial activity against a pulmonary infection with the P. aeruginosa strain of serotype G. Surprisingly, when a neutropenic mouse model of systemic infection with P. aeruginosa identified by the P. aeruginosa strain of serotype G (ATCC 27584) was used and comparison was made by using Venilon as a control, the ED50 value of antibacterial effect of each of these antibodies was 1/100 or less of the ED50 value of Venilon. Particularly, for a neutropenic mouse model of systemic infection with a P. aeruginosa strain identified by ATCC 27584, the antibody “1584” exhibited such an excellent effect that the ED50 value of the antibody “1584” was 1/300 or less of the ED50 value of Venilon. Accordingly, when a systemic infection mouse model is used, the ED50 value of the anti-serotype G LPS antibody of the present invention is preferably 1/100 or less (for example, 1/150 or less, 1/200 or less, 1/250 or less or 1/300 or less) of that of Venilon.

The anti-serotype G LPS antibody of the present invention can have any one of the above-described activities alone, but preferably has multiple activities together.

Another preferred embodiment of the anti-serotype G LPS antibody of the present invention is an antibody comprising a light chain variable region including light chain CDRs 1 to 3 and a heavy chain variable region including heavy chain CDRs 1 to 3, of the antibody (1584, 1573, 1572 or 1587) identified in the present invention. Specific examples thereof include the following antibodies (i) to (iv):

(i) an antibody comprising a light chain variable region including light chain CDRs 1 to 3 (amino acid sequences described in SEQ ID NOs: 1 to 3) and a heavy chain variable region including heavy chain CDRs 1 to 3 (amino acid sequences described in SEQ ID NOs: 4 to 6), for example, an antibody in which a light chain variable region includes an amino acid sequence described in SEQ ID NO: 7 and a heavy chain variable region includes an amino acid sequence described in SEQ ID NO: 8;

(ii) an antibody comprising a light chain variable region including light chain CDRs 1 to 3 (amino acid sequences described in SEQ ID NOs: 9 to 11) and a heavy chain variable region including heavy chain CDRs 1 to 3 (amino acid sequences described in SEQ ID NOs: 12 to 14), for example, an antibody in which a light chain variable region includes an amino acid sequence described in SEQ ID NO: 15 and a heavy chain variable region includes an amino acid sequence described in SEQ ID NO: 16;

(iii) an antibody comprising a light chain variable region including light chain CDRs 1 to 3 (amino acid sequences described in SEQ ID NOs: 17 to 19) and a heavy chain variable region including heavy chain CDRs 1 to 3 (amino acid sequences described in SEQ ID NOs: 20 to 22), for example, an antibody in which a light chain variable region includes an amino acid sequence described in SEQ ID NO: 23 and a heavy chain variable region includes an amino acid sequence described in SEQ ID NO: 24; and

(iv) an antibody comprising a light chain variable region including light chain CDRs 1 to 3 (amino acid sequences described in SEQ ID NOs: 25 to 27) and a heavy chain variable region including heavy chain CDRs 1 to 3 (amino acid sequences described in SEQ ID NOs: 28 to 30), for example, an antibody in which a light chain variable region includes an amino acid sequence described in SEQ ID NO: 31 and a heavy chain variable region includes an amino acid sequence described in SEQ ID NO: 32.

The present invention also provides a peptide comprising any one of a light chain, a heavy chain and variable regions thereof, of an antibody, the peptide comprising CDR identified in the antibody (1584, 1573, 1572 or 1587) of the present invention.

Examples of a peptide comprising anyone of a light chain, a heavy chain and variable regions thereof, of an antibody, the peptide comprising CDR of the antibody 1584, include the following peptides (i) and (ii):

(i) a peptide comprising a light chain or a light chain variable region of the antibody of the present invention, the peptide comprising the amino acid sequences described in SEQ ID NOs: 1 to 3, for example, a peptide comprising the amino acid sequence described in SEQ ID NO: 7; and

(ii) a peptide comprising a heavy chain or a heavy chain variable region of the antibody of the present invention, the peptide comprising the amino acid sequences described in SEQ ID NOs: 4 to 6, for example, a peptide comprising the amino acid sequence described in SEQ ID NO: 8.

Examples of a peptide comprising anyone of alight chain, a heavy chain and variable regions thereof, of an antibody, the peptide comprising CDR of the antibody 1573, include the following peptides (i) and (ii):

(i) a peptide comprising a light chain or a light chain variable region of the antibody of the present invention, the peptide comprising the amino acid sequences described in SEQ ID NOs: 9 to 11, for example, a peptide comprising the amino acid sequence described in SEQ ID NO: 15; and

(ii) a peptide comprising a heavy chain or a heavy chain variable region of the antibody of the present invention, the peptide comprising the amino acid sequences described in SEQ ID NOs: 12 to 14, for example, a peptide comprising the amino acid sequence described in SEQ ID NO: 16.

Examples of a peptide comprising anyone of a light chain, a heavy chain and variable regions thereof, of an antibody, the peptide comprising CDR of the antibody 1572, include the following peptides (i) and (ii):

(i) a peptide comprising a light chain or a light chain variable region of the antibody of the present invention, the peptide comprising the amino acid sequences described in SEQ ID NOs: 17 to 19, for example, a peptide comprising the amino acid sequence described in SEQ ID NO: 23; and

(ii) a peptide comprising a heavy chain or a heavy chain variable region of the antibody of the present invention, the peptide comprising the amino acid sequences described in SEQ ID NOs: 20 to 22, for example, a peptide comprising the amino acid sequence described in SEQ ID NO: 24.

Examples of a peptide comprising anyone of a light chain, a heavy chain and variable regions thereof, of an antibody, the peptide comprising CDR of the antibody 1587, include the following peptides (i) and (ii):

(i) a peptide comprising a light chain or a light chain variable region of the antibody of the present invention, the peptide comprising the amino acid sequences described in SEQ ID NOs: 25 to 27, for example, a peptide comprising the amino acid sequence described in SEQ ID NO: 31; and

(ii) a peptide comprising a heavy chain or a heavy chain variable region of the antibody of the present invention, the peptide comprising the amino acid sequences described in SEQ ID NOs: 28 to 30, for example, a peptide comprising the amino acid sequence described in SEQ ID NO: 32.

A functional antibody can be prepared by linking such peptides with, for example, a linker.

Once a specific anti-serotype G LPS antibody (1584, 1573, 1572 or 1587) is obtained, those skilled in the art can identify an epitope recognized by the antibody, and prepare various antibodies which bind to the epitope. The present invention also provides an antibody which recognizes an epitope identical to that recognized by any one of the antibody “1584,” the antibody “1573,” the antibody “1572” and the antibody “1587.” It is conceivable that such an antibody has the above-described characteristics of the one of the antibody “1584,” the antibody “1573,” the antibody “1572” and the antibody “1587” (the serotype specificity of binding activity to P. aeruginosa, the opsonic activity, the agglutination activity, and the antibacterial activity against a systemic infection).

The binding of an antibody to P. aeruginosa can be evaluated, for example, by the Whole cell ELISA method, as described in the examples of the present application. Thereby, the range of serotypes of P. aeruginosa strains to which the antibody exhibits a binding activity can be determined. The opsonic activity can be evaluated, for example, by the detection method using, as an index, a fluorescence intensity of human polymorphonuclear leukocytes incorporating FITC-labeled P. aeruginosa, as described in the examples of the present application. Meanwhile, the agglutination activity can be evaluated, for example, as an agglutination titer per amount of IgG, by detecting an agglutinating ability of an antibody against serially diluted bacterial cells, as described in the examples of the present application. Meanwhile, the antibacterial activity against a systemic infection can be evaluated, for example, from a survival rate of model mice to which an antibody is administered, as described in the examples of the present application.

The antibody of the present invention is typically a human antibody. However, by using information on the epitopes identified in the present invention or by using CDR regions or variable regions of the human antibodies identified in the present invention, those skilled in the art can prepare various antibodies such as, for example, chimeric antibodies, humanized antibody and mouse antibodies, in addition to human antibodies, and also can prepare functional fragments of these antibodies. For administration to humans as a medicine, the antibody of the present invention is most preferably a human antibody, from the viewpoint of side effect reduction.

In the present invention, a “human antibody” refers to an antibody of which all regions are originated from human. For the preparation of a human antibody, the methods described in the present examples can be employed. As other methods, for example, a method can be used in which a transgenic animal (for example, a mouse) capable of producing a repertoire of human antibodies by immunization is used. Preparation methods of such human antibodies have been known (for example, Nature, 362: 255-258 (1992), Intern. Rev. Immunol, 13: 65-93 (1995), J. Mol. Biol, 222: 581-597 (1991), Nature Genetics, 15: 146-156 (1997), Proc. Natl. Acad. Sci. USA, 97: 722-727 (2000), Japanese Unexamined Patent Application Publication No. Hei 10-146194, Japanese Unexamined Patent Application Publication No. Hei 10-155492, Japanese Patent No. 2938569, Japanese Unexamined Patent Application Publication No. Hei 11-206387, and International Application Japanese-Phase Publication No. Hei 8-509612, and International Application Japanese-Phase Publication No. Hei 11-505107).

In the present invention, a “chimeric antibody” refers to an antibody obtained by linking a variable region of an antibody of one species with a constant region of an antibody of another species. For example, such a chimeric antibody can be obtained as follows. A mouse is immunized with an antigen. A portion coding an antibody variable part (variable region) which binds to the antigen is cut out from a gene coding a monoclonal antibody of the mouse. The portion is linked with a gene coding a human bone marrow-derived antibody constant part (constant region). These linked genes are incorporated in an expression vector. The expression vector is then introduced into a host which produces a chimeric antibody (Refer to, for example, Japanese Unexamined Patent Application Publication No. Hei 8-280387, U.S. Pat. No. 4,816,397, U.S. Pat. No. 4,816,567, and U.S. Pat. No. 5,807,715). Meanwhile, in the present invention, a “humanized antibody” refers to an antibody obtained by grafting a genome sequence of an antigen-binding site (CDR) of a non-human-derived antibody onto a gene of a human antibody (CDR grafting). Preparation methods of such chimeric antibodies have been known (refer to, for example, EP239400, EP125023, WO90/07861, and WO96/02576). In the present invention, a “functional fragment” of an antibody means a part (a partial fragment) of an antibody, which retains a capability of specifically recognizing an antigen of the antibody from which the part is originated. Specific examples of the functional fragment include Fab, Fab′, F(ab′)2, a variable region fragment (Fv), a disulfide-linked Fv, a single-chain Fv (scFv), sc(Fv)2, a diabody, a polyspecific antibody, and polymers thereof.

Here, the “Fab” means a monovalent antigen-binding fragment, of a immunoglobulin, formed of a part of one light chain and a part of one heavy chain. The Fab can be obtained by papain-digestion of an antibody, or a recombinant method. The “Fab′” differs from the Fab in that, in Fab′, a small number of residues including one or more cysteines from a hinge region of an antibody are added to the carboxy terminus of a heavy chain CH1 domain. The “F(ab′)2” means a divalent antigen-binding fragment, of an immunoglobulin, made of parts of both light chains and parts of both heavy chains.

The “variable region fragment (Fv)” is a smallest antibody fragment which has a complete antigen recognition and binding site. The Fv is a dimer in which a heavy chain variable region and a light chain variable region are strongly linked by non-covalent bonding. The “single-chain Fv (scFv)” includes a heavy chain variable region and a light chain variable region of an antibody, and in the “single-chain Fv (scFv),” these regions exist in a single polypeptide chain. The “sc(Fv)2” is a single chain obtained by bonding two heavy chain variable regions and two light chain variable regions with a linker or the like. The “diabody” is a small antibody fragment having two antigen binding sites. The fragment include a heavy chain variable region bonded to a light chain variable region in a single polypeptide chain, and each of the regions forms a pair with a complementary region in another chain. The “polyspecific antibody” is a monoclonal antibody which has binding specificity to at least two different antigens. For example, such a polyspecific antibody can be prepared by coexpression of two immunoglobulin heavy chain/light chain pairs, in which two heavy chains have mutually different specificities.

The antibody of the present invention includes antibodies whose amino acid sequences are modified without impairing desirable activities (the binding activity to P. aeruginosa and the broadness thereof or the specificity thereof, the opsonic activity, the agglutination activity, the antibacterial activity against a systemic infection or a pulmonary infection, and/or other biological characteristics). An amino acid sequence variant of the antibody of the present invention can be prepared by introduction of mutation into a DNA coding an antibody chain of the present invention or by peptide synthesis. Such modification includes, for example, substitution, deletion, addition and/or insertion of one or multiple residues in an amino acid sequence of the antibody of the present invention. The modification region of the amino acid sequence of the antibody may be a constant region of a heavy chain or a light chain of the antibody or a variable region (a framework region or CDR) thereof, as long as the resulting antibody has activities which are equivalent to those of an unmodified antibody. It is conceivable that modification on amino acids other than those in CDR has a relatively small effect on binding affinity for an antigen. As of now, there are screening methods of antibodies whose affinity for an antigen is enhanced by modification of amino acids in CDR(PNAS, 102: 8466-8471 (2005), Protein Engineering, Design & Selection, 21: 485-493 (2008), International Publication No. WO2002/051870, J. Biol. Chem., 280: 24880-24887 (2005), and Protein Engineering, Design & Selection, 21: 345-351 (2008)).

The number of amino acids modified are preferably 10 amino acids or less, more preferably 5 amino acids or less, and most preferably 3 amino acids or less (for example, 2 amino acids or less, or 1 amino acid). The modification of amino acids is preferably conservative substitution. In the present invention, the term “conservative substitution” means substitution with a different amino acid residue having a chemically similar side chain. Groups of amino acids having chemically similar amino acid side chains are well known in the technical field to which the present invention pertains. For example, amino acids can be grouped into acidic amino acids (aspartic acid and glutamic acid), basic amino acids (lysine, arginine, and histidine), and neutral amino acids. The neutral amino acids can be sub-classified into amino acids having a hydrocarbon group (glycine, alanine, valine, leucine, isoleucine and proline), amino acids having a hydroxy group (serine and threonine), sulfur-containing amino acids (cysteine and methionine), amino acids having an amide group (asparagine and glutamine), an amino acid having an imino group (proline); and amino acids having an aromatic group (phenylalanine, tyrosine and tryptophan).

The modification on the antibody of the present invention may be modification on post-translational process of the antibody, for example, the change in number of sites of glycosylation or in location of the glycosylation. This can improve, for example, an ADCC activity of the antibody. Glycosylation of an antibody is typically N-linked or O-linked glycosylation. The glycosylation of an antibody greatly depends on a host cell used for expression of the antibody. Alteration in glycosylation pattern can be performed by a known method such as introduction or deletion of a certain enzyme which is related to carbohydrate production (Japanese Unexamined Patent Application Publication No. 2008-113663, U.S. Pat. No. 5,047,335, U.S. Pat. No. 5,510,261, U.S. Pat. No. 5,278,299, International Publication No. WO99/54342). In the present invention, for the purpose of increasing the stability of an antibody or other purposes, an amino acid subjected to deamidation or an amino acid which is adjacent to an amino acid subjected to deamidation may be substituted with a different amino acid to prevent the deamidation. Moreover, a glutamic acid can be substituted with a different amino acid to thereby increase the stability of an antibody. The present invention also provides an antibody thus stabilized.

The polyclonal antibody of the antibodies of the present invention can be obtained as follows. Specifically, an immune animal is immunized with an antigen (LPS, a molecule having a partial structure of LPS, P. aeruginosa on which surface any one of LPS and a molecule having a partial structure of LPS is exposed, or the like). A polyclonal antibody can be obtained by purification of an antiserum obtained from the animal by a conventional method (for example, salting-out, centrifugation, dialysis, column chromatography, or the like). Meanwhile, the monoclonal antibody can be prepared by a standard hybridoma method or a standard recombinant DNA method, in addition to the methods described in the present examples.

A typical example of the hybridoma method is a Kohler & Milstein method (Kohler & Milstein, Nature, 256: 495 (1975)). Antibody-producing cells used in cell fusion process of this method are spleen cells, lymph node cells, peripheral blood leukocytes, and the like of an animal (for example, mouse, rat, hamster, rabbit, monkey or goat) which is immunized with an antigen (LPS, a molecule having a partial structure of LPS, P. aeruginosa on which surface any of LPS and a molecule having a partial structure of LPS is exposed, or the like). Antibody-producing cells obtained by causing an antigen to act, in a culture medium, on any of cells of the above described types and lymphocytes which are isolated from a non-immunized animal in advance can be used. As the myeloma cells, various known cell strains can be used. The antibody-producing cells and the myeloma cells may be originated from different animal species, as long as the antibody-producing cells and the myeloma cells can be fused. However, the antibody-producing cells and the myeloma cells are preferably originated from the same animal species. Hybridomas can be produced by, for example, by cell fusion between spleen cells obtained from a mouse immunized with an antigen and mouse myeloma cells. Thereafter, by screening the hybridomas, a hybridoma which produces a LPS antigen-specific monoclonal antibody can be obtained. The monoclonal antibody against a LPS antigen can be obtained by culturing the hybridoma, or from the ascites in a mammal to which the hybridoma is administered.

The recombinant DNA method is a method with which the above-described antibody of the present invention is produced as a recombinant antibody as follows. A DNA coding the antibody or the peptide of the present invention is cloned from a hybridoma, B cells, or the like. The cloned DNA is incorporated in an appropriate vector, and the vector is introduced into host cells (for example, a mammalian cell strain, Escherichia coli, yeast cells, insect cells, plant cells, or the like) (for example, P. J. Delves, Antibody Production: Essential Techniques, 1997 WILEY, P. Shepherd and C. Dean Monoclonal Antibodies, 2000 OXFORD UNIVERSITY PRESS, Vandamme A. M. et al., Eur. J. Biochem. 192: 767-775 (1990)). For the expression of a DNA cording the antibody of the present invention, DNAs coding a heavy chain and a light chain may be incorporated in expression vectors, respectively, and host cells may be transformed. Alternatively, DNAs coding a heavy chain and a light chain may be incorporated in a single expression vector, and host cells may be transformed (refer to WO94/11523). The antibody of the present invention can be obtained in a substantially pure and homogeneous form by culturing of the above-described host cells, and separation and purification from the host cells or a culture medium. For the separation and purification of the antibody, any method used for standard purification of polypeptide can be used. When a transgenic animal (cattle, goat, sheep, pig or the like) in which an antibody gene is incorporated is produced by a transgenic animal production technique, a large amount of a monoclonal antibody derived from the antibody gene can also be obtained from milk of the transgenic animal.

The present invention also provides a DNA coding the above-described antibody or peptide of the present invention, a vector containing the DNA, host cells having the DNA, and a method of producing an antibody, the method including culturing the host cell and collecting an antibody.

Since the antibody of the present invention has the above-described activities, the antibody of the present invention can be used for prevention or treatment of Diseases associated with P. aeruginosa. Accordingly, the present invention also provides a pharmaceutical composition for use in prevention or treatment of a disease associated with P. aeruginosa, the pharmaceutical composition comprising the antibody of the present invention as an active ingredient, and a method for preventing or treating a disease associated with P. aeruginosa, comprising a step of administering a therapeutically or preventively effective amount of the antibody of the present invention to a mammal including a human. The treatment or prevention method of the present invention can be used for various mammals, in addition to humans, including, for example, dogs, cats, cattle, horses, sheep, pigs, goats, and rabbits.

Examples of the disease associated with P. aeruginosa include systemic infectious diseases, caused by a P. aeruginosa infection including a multidrug resistant P. aeruginosa infection, for example, septicemia, meningitis, and endocarditis. Other examples thereof include: otitis media and sinusitis in the otolaryngologic field; pneumonia, chronic respiratory tract infection, and catheter infection in the pulmonary field; postoperative peritonitis and postoperative infection in a biliary tract or the like in the surgical field; abscess of eyelid, dacryocystitis, conjunctivitis, corneal ulcer, corneal abscess, panophthalmitis, and orbital infection in the ophthalmological field; and urinary tract infections including complicated urinary tract infection, catheter infection, and abscess around the anus in the urologic field. Besides, the examples include burns (including a serious burn and a burn of the respiratory tract), decubital infection, and cystic fibrosis.

A pharmaceutical composition or an agent of the present invention may be used in the form of a composition which uses the antibody of the present invention as an active ingredient, and preferably which contains a purified antibody composition and another component, for example, saline, an aqueous glucose solution or a phosphate buffer.

The pharmaceutical composition of the present invention may be formulated into a preparation in a liquid or lyophilized form as necessary, and may optionally comprise a pharmaceutically acceptable carrier, for example, a stabilizer, a preservative, and an isotonic agent. Examples of the pharmaceutically acceptable carrier includes: mannitol, lactose, saccharose, and human albumin for a lyophilized preparation; and saline, water for injection, a phosphate buffer, and aluminum hydroxide for a liquid preparation. However, the examples are not limited thereto.

An administration may differ depending on the age, weight, gender, and general health state of an administration target. The administration can be carried out by any administration route of oral administration and parenteral administration (for example, intravenous administration, intraarterial administration, and local administration). However, parenteral administration is preferable.

The dose of the pharmaceutical composition varies depending on the age, weight, sex, and general health state of a patient, the severity of a P. aeruginosa infection and components of an antibody composition to be administered. The dose of the antibody composition of the present invention is generally 0.1 to 1000 mg, and preferably 1 to 100 mg, per kg body weight per day for an adult in a case of intravenous administration.

The pharmaceutical composition of the present invention is preferably administered in advance to a patient who may develop a P. aeruginosa infection.

Since the antibody of the present invention binds to LPS exposed on the cell surface of P. aeruginosa, the antibody of the present invention can also be used as a P. aeruginosa infection diagnostic agent.

When the antibody of the present invention is prepared as a diagnostic agent, the diagnostic agent can be obtained in any dosage form by adopting any means suitable for the purpose. For example, ascites, a culture medium containing an antibody of interest, or a purified antibody is measured for the antibody titer and appropriately diluted with PBS (phosphate buffer containing saline) or the like; thereafter, a preservative such as 0.1% sodium azide is added thereto. Alternatively, the antibody of the present invention adsorbed to latex or the like is determined for the antibody titer and appropriately diluted, and a preservative is added thereto for use. The antibody of the present invention bound to latex particles as described above is one of preferable dosage forms as a diagnostic agent. As the latex in this case, appropriate resin materials, for example, latex of polystyrene, polyvinyl toluene, or polybutadiene, are suitable.

According to the present invention, provided is a diagnosis method for a P. aeruginosa infection using the antibody of the present invention. The diagnosis method of the present invention can be carried out by collecting a biological sample such as expectoration, a lung lavage fluid, pus, a tear, blood, or urine from mammals, including a human, which may have developed a P. aeruginosa infection, subsequently bringing the collected sample into contact with the antibody of the present invention, and determining whether or not an antigen-antibody reaction occurs.

According to the present invention, provided is a kit for detecting the presence of P. aeruginosa, the kit comprising at least the antibody of the present invention.

The antibody of the present invention may be labeled. This kit for detection detects the presence of P. aeruginosa by detecting the antigen-antibody reaction.

Thus, the detection kit of the present invention can further include various reagents for carrying out the antigen-antibody reaction, for example, a secondary antibody, a chromogenic reagent, a buffer, instructions, and/or an instrument used in an ELISA method, and the like, if desired.

### EXAMPLES

Hereinafter, the present invention will be described more specifically on the basis of examples. However, the present invention is not limited to these examples.

### Example 1

### Cloning of Anti-LPS Antibody

### (1) Blood Donor Recruitment

250 ml blood samples were collected from Cystic Fibrosis Patients having a chronic PA lung infection and from healthy volunteers. Donors were generally of good health and represented a wide range in age, years of chronic PA infection, as well as immune response status. Additional inclusion criteria were an age above 18 years, a body weight above 50 kilograms and normal hemoglobin levels. All donations were approved by the Danish National Committee on Biomedical Research Ethics.

The following types of analyses were performed on each blood samples: i) FACS analyses to determine the amount of circulating plasma blasts and plasma cells, ii) ELISPOT analyses to determine the amount of circulating antibody producing cells specific for particular LPS antigens, iii) ELISA analyses to determine the presence of specific immunoglobulin towards particular LPS antigens.

Donor samples with a high percentage of plasma blasts specific for LPS antigens were chosen for the Symplex procedure (refer to WO2005/042774) described below.

### (2) FACS Sorting of Human Plasmablasts

The starting materials for this procedure were MACS-purified CD19 positive B-cells. These cells were normally stored frozen and then a fraction was thawed before each sorting.

Viable plasma blasts were identified by staining cells for CD19,

CD38, the lambda-light chain and dead cells.

Freshly thawed cells were washed twice with 4 ml FACS PBS, diluted to 1×10⁶ cells per 40 μl FACS PBS. Per 1×10⁶ cells the following reagents was added: 10 μl CD19-FITC, 20 μl CD38 APC and 10 μl Lambda-PE at 4° C. and left for 20 minutes in the dark on ice. Samples were washed twice with 2 ml FACS buffer and resuspended in 1 ml FACS PBS whereafter propidium iodide was added (1:100). The cell-suspension was filtered through a 50 μm Syringe falcon (FACS filter), and was ready for sorting directly into Symplex PCR plates (see next section). After sorting, PCR plates were centrifuged at 300×g for 1 minutes and stored at −80° C. for later use.

### (3) Linkage of Cognate VH and VL Pairs

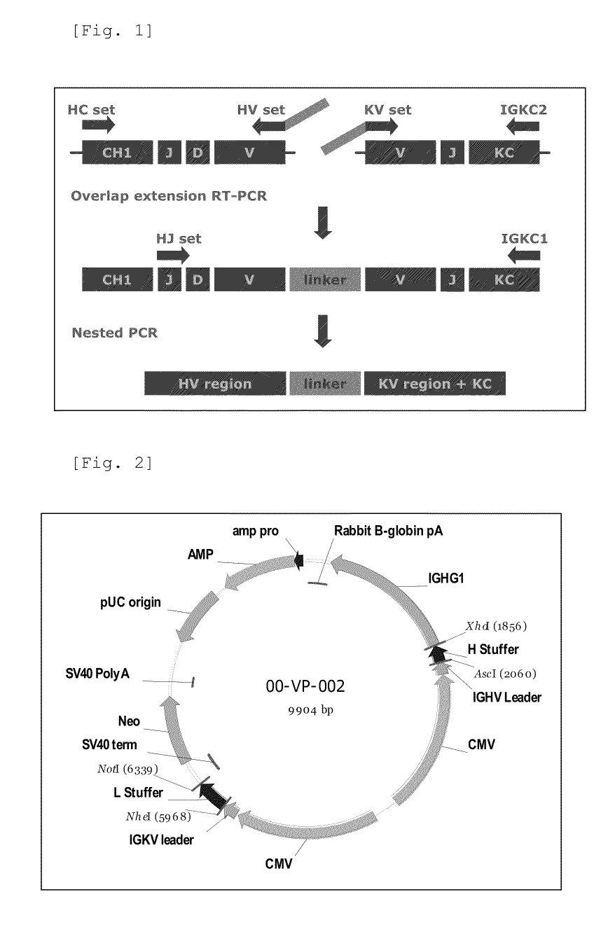

In order to pair sequences coding a heavy chain variable region (VH) and a light chain variable region (VL) which were originated form the same B cell, the sequences coding the VH and the VL were linked on a single cell gated as plasma cells. The procedure utilized a two step PCR procedure based on a one-step multiplex overlap-extension RT-PCR followed by a nested PCR. The primer mixes used in the present example only amplify Kappa light chains. The principle for linkage of cognate VH and VL sequences was showed in FIG. 1.

The 96-well PCR plates produced were thawed and the sorted cells served as template for the multiplex overlap-extension RT-PCR. The sorting buffer added to each well before the single-cell sorting contained reaction buffer (OneStep RT-PCR Buffer; Qiagen), primers for RT-PCR (refer to Table 2) and RNase inhibitor (RNasin, Promega). This was supplemented with OneStep RT-PCR Enzyme Mix (25× dilution; Qiagen) and dNTP mix (200 μM each) to obtain the given final concentration in a 20-μl reaction volume.

TABLE 2

| | | |
|---|---|---|
| Symplex ™ | | Final |
| primer | | concentration |
| mix | Sequence (5′-3′) | (pmol/μL) |

Multiplex PCR

| | | |
|---|---|---|
| KC | | |
| IGKC2 | ATATATATGCGGCCGCTTATTAACACTCTCCCCTGTTG | 51.25 |
| | (SEQ ID NO: 41) | |
| HC set | | |
| IGHG | GACSGATGGGCCCTTGGTGG | 51.25 |
| | (SEQ ID NO: 42) | |
| IGHA | GAGTGGCTCCTGGGGGAAGA | 51.25 |
| | (SEQ ID NO: 43) | |
| HV set | | |
| HV1 | TATTCCCATGGCGCGCCCAGRTGCAGCTGGTGCART | 10.24 |
| | (SEQ ID NO: 44) | |
| HV2 | TATTCCCATGGCGCGCCSAGGTCCAGCTGGTRCAGT | 10.24 |
| | (SEQ ID NO: 45) | |
| HV3 | TATTCCCATGGCGCGCCCAGRTCACCTTGAAGGAGT | 10.24 |
| | (SEQ ID NO: 46) | |
| HV4 | TATTCCCATGGCGCGCCSAGGTGCAGCTGGTGGAG | 10.24 |
| | (SEQ ID NO: 47) | |
| HV5 | TATTCCCATGGCGCGCCCAGGTGCAGCTACAGCAGT | 10.24 |
| | (SEQ ID NO: 48) | |
| HV6 | TATTCCCATGGCGCGCCCAGSTGCAGCTGCAGGAGT | 10.24 |
| | (SEQ ID NO: 49) | |
| HV7 | TATTCCCATGGCGCGCCGARGTGCAGCTGGTGCAGT | 10.24 |
| | (SEQ ID NO: 50) | |
| HV8 | TATTCCCATGGCGCGCCCAGGTACAGCTGCAGCAGTC | 10.24 |
| | (SEQ ID NO: 51) | |
| KV set | | |
| KV1 | GGCGCGCCATGGGAATAGCTAGCCGACATCCAGWTGACCCAGTCT | 10.24 |
| | (SEQ ID NO: 52) | |
| KV2 | GGCGCGCCATGGGAATAGCTAGCCGATGTTGTGATGACTCAGTCT | 10.24 |
| | (SEQ ID NO: 53) | |
| KV3 | GGCGCGCCATGGGAATAGCTAGCCGAAATTGTGWTGACRCAGTCT | 10.24 |
| | (SEQ ID NO: 54) | |
| KV4 | GGCGCGCCATGGGAATAGCTAGCCGATATTGTGATGACCCACACT | 10.24 |
| | (SEQ ID NO: 55) | |
| KV5 | GGCGCGCCATGGGAATAGCTAGCCGAAACGACACTCACGCAGT | 10.24 |
| | (SEQ ID NO: 56) | |
| KV6 | GGCGCGCCATGGGAATAGCTAGCCGAAATTGTGCTGACTCAGTCT | 10.24 |
| | (SEQ ID NO: 57) | |

Nested PCR

| | | |
|---|---|---|
| KC | | |
| IGKC1 | ACCGCCTCCACCGGCGGCCGCTTATTAACACTCTCCCCTGTTGAAGCTCTT | 51.25 |
| | (SEQ ID NO: 58) | |
| HJ set | | |
| IGHJ 1-2 | GGAGGCGCTCGAGACGGTGACCAGGGTGCC | 51.25 |
| | (SEQ ID NO: 59) | |
| IGHJ 3 | GGAGGCGCTCGAGACGGTGACCATTGTCCC | 51.25 |
| | (SEQ ID NO: 60) | |
| IGHJ 4-5 | GGAGGCGCTCGAGACGGTGACCAGGGTTCC | 51.25 |
| | (SEQ ID NO: 61) | |
| IGHJ 6 | GGAGGCGCTCGAGACGGTGACCGTGGTCCC | 51.25 |
| | (SEQ ID NO: 62) | |

The plates were incubated for 30 minutes at 55° C. to allow for reverse transcription of the RNA from each cell. After the reverse transcription, the plates were subjected to the following PCR cycle: 10 minutes at 94° C., 35×(40 seconds at 94° C., 40 seconds at 60° C., 5 minutes at 72° C.), 10 minutes at 72° C.

The PCR reactions were performed in H20BIT Thermal cycler (ABgene) with a Peel Seal Basket for 24 96-well plates to facilitate a high-throughput. The PCR plates were stored at −20° C. after cycling.

For the nested PCR step, 96-well PCR plates were prepared with the following mixture in each well (20-μl reactions) to obtain the given final concentration: 1× FastStart buffer (Roche), dNTP mix (200 μM each), nested primer mix (see Table 1), Phusion DNA Polymerase (0.08 U; Finnzymes) and FastStart High Fidelity Enzyme Blend (0.8 U; Roche). As template for the nested PCR, 1 μl was transferred from the multiplex overlap-extension PCR reactions. The nested PCR plates were subjected to the following thermo cycling: 35×(30 seconds at 95° C., 30 seconds at 60° C., 90 seconds at 72° C.), 10 minutes at 72° C.

Randomly selected reaction products were analyzed on a 1% agarose gel to verify the presence of an overlap-extension fragment of approximately 1050 base pairs (bp). The plates were stored at −20° C. until further processing of the PCR fragments. The repertoires of linked VH and VL coding pairs from the nested PCR were pooled, without mixing pairs from different donors, and were purified by preparative 1% agarose gel electrophoresis.

(4) Insertion of Cognate VH and VL Coding Sequence Pairs into a Screening Vector

In order to identify antibodies with binding specificity to LPS, the VH and VL coding sequences obtained were expressed as full-length antibodies. This involved insertion of the repertoire of VH and VL coding pairs into an expression vector and transfection into a host cell.

A two-step cloning procedure was employed for generation of a repertoire of expression vectors containing the linked VH and VL coding pairs. Statistically, if the repertoire of expression vectors contains ten times as many recombinant plasmids as the number of cognate paired VH and VL PCR products used for generation of the screening repertoire, there is 99% likelihood that all unique gene pairs are represented. Thus, if 400 overlap-extension V-gene fragments were obtained, a repertoire of at least 4000 clones was generated for screening.

Briefly, the purified PCR product of the repertoires of linked VH and VL coding pairs were cleaved with XhoI and NotI

DNA endonucleases at the recognition sites introduced into the termini of PCR products. The cleaved and purified fragments were ligated into an XhoI/NotI digested mammalian IgG expression vector, OO-VP-002 (FIG. 2) by standard ligation procedures. The ligation mix was electroporated into E. coli and added to 2xYT plates containing the appropriate antibiotic and incubated at 37° C. over night. The amplified repertoire of vectors was purified from cells recovered from the plates using standard DNA purification methods (Qiagen).

The plasmids were prepared for insertion of promoter-leader fragments by cleavage using AscI and NheI endonucleases. The restriction sites for these enzymes were located between the VH and VL coding gene pairs. Following purification of the vector, an AscI-NheI digested bi-directional mammalian promoter-leader fragment was inserted into the AscI and NheI restriction sites by standard ligation procedures. The ligated vector was amplified in E. coli and the plasmid was purified using standard methods. The generated repertoire of screening vectors was transformed into E. coli by conventional procedures. Colonies obtained were consolidated into 384-well master plates and stored. The number of colonies transferred to the 384-well plates exceeded the number of used PCR products by at least 3-fold, thus giving 95% likelihood for presence of all unique V-gene pairs obtained.

M166 was expressed as a chimeric IgG antibody. The variable gene amino acid sequences of M166 originate from a murine antibody specific for the Pseudomonas aeruginosa PcrV protein as described in the patent WO2002/064161. Variable genes were synthesized at GENEART AG (BioPark, Josef-Engert-Str. 11, 93053 Regensburg, Germany) and in that process linking the murine light chain variable gene to the human kappa constant gene. The murine heavy chain variable gene and the chimeric light chain gene were inserted into an expression vector harboring the remaining part of the human heavy chain constant genes as well as elements required for gene expression in mammalian cells.

### (5) Expression of Symplex Repertoires

The bacteria colonies on the master plates were planted in a culture medium in 384-well plates, and cultured overnight. A DNA for transfection was prepared from each well using TempliPhi DNA amplification Kit (Amersham Biosciences) in accordance of the manual thereof. On the day before the transfection, Flp-In™-CHO cells (Invitrogen) were planted in the 384-well plates at 3000 cells per well (in 20 μl of culture medium). The amplified DNAs were introduced into cells using FuGENE 6 (Roche) in accordance with the manual thereof. After 3-day culture, the supernatant containing full-length antibodies was collected, and stored for antigen specificity screening.

### (6) Screening for Binding to LPS

By an ELISA method, screening of antibody library was performed using the binding to a mixture of purified LPS molecules isolated from related P. aeruginosa type strains as an index. A Nunc MaxiSorp 384-well plate was coated at 4° C. overnight with a LPS mixture (containing 6 serotypes per assay at maximum) obtained by diluting a mixture of purified LPS molecules with a 50 mM carbonate buffer (pH: 9.6) so that 10 μg/ml of purified LPS of each LPS serotype was contained. The well plate was blocked by 50 μl of PBS-T (PBS+0.05% Tween) containing 2% of skimmed milk (SM), and then washed once with PBS-T. 15 μl of an antibody supernatant was added into each well and incubation at room temperature for 1.5 hours was performed. Then, the plate was washed once with PBS-T. To detect antibodies binding to the wells, a secondary antibody (HRP-Goat-anti-human IgG, Jackson) diluted 10.000-fold with 2% SM-PBS-T was added to each well, then incubation was performed at room temperature for 1 hour. The plate was washed once with PBS-T, and then 25 μl of a substrate (Kemen-tec Diagnostics, catalog No. 4390) was added to each well. Then, incubation was performed for 5 minutes. After the incubation, 25 μl of 1 M sulfuric acid was added to terminate the reaction. A specific signal was detected by 450 nm-ELISA reader.

### (7) Sequence Analysis and Clone Selection

The clones identified as LPS-specific in ELISA were retrieved from the original master plates (384-well format) and consolidated into new plates. DNA was isolated from the clones and submitted for DNA sequencing of the V-genes. The sequences were aligned and all the unique clones were selected. Multiple alignments of obtained sequences revealed the uniqueness of each particular clone and allowed for identification of unique antibodies. Multiple genetically distinct antibody sequence clusters were identified. Each cluster of related sequences have probably been derived through somatic hypermutations of a common precursor clone. Overall, one to two clones from each cluster was chosen for validation of sequence and specificity.

### (8) Sequence and Specificity Validation

In order to validate the antibody encoding clones, DNA plasmid was prepared and transfection of FreeStyle CHO-S cells (Invitrogen) in 2-ml scale was performed for expression. The supernatant were harvested 96 hours after transfection. Expression levels were estimated with standard anti-IgG ELISA, and the specificity was determined by LPS-specific ELISA.

### (9) Identified Antibody

As a result of the above, identified anti-LPS antibodies and the sequences of CDRs and variable regions of the identified anti-LPS antibodies are as follows. Note that the sequences of constant regions of the identified anti-LPS antibodies are as described in WO 2005/042774.

### <Anti-Serotype G LPS Antibody>

“1584”
SEQ ID NOs: 1 to 3••amino acid sequences of light chain CDRs 1 to 3
SEQ ID NOs: 4 to 6••amino acid sequences of heavy chain CDRs 1 to 3
SEQ ID NO: 7••an amino acid sequence of a light chain variable region
SEQ ID NO: 8••an amino acid sequence of a heavy chain variable region
SEQ ID NO: 33••a base sequence of a light chain variable region
SEQ ID NO: 34••a base sequence of a heavy chain variable region
“1573”
SEQ ID NOs: 9 to 11••amino acid sequences of light chain CDRs 1 to 3
SEQ ID NOs: 12 to 14••amino acid sequences of heavy chain CDRs 1 to 3
SEQ ID NO: 15••an amino acid sequence of a light chain variable region
SEQ ID NO: 16••an amino acid sequence of a heavy chain variable region
SEQ ID NO: 35••a base sequence of a light chain variable region
SEQ ID NO: 36••a base sequence of a heavy chain variable region
“1572”
SEQ ID NOs: 17 to 19••amino acid sequences of light chain CDRs 1 to 3
SEQ ID NOs: 20 to 22••amino acid sequences of heavy chain CDRs 1 to 3
SEQ ID NO: 23••an amino acid sequence of a light chain variable region
SEQ ID NO: 24••an amino acid sequence of a heavy chain variable region
SEQ ID NO: 37••a base sequence of a light chain variable region
SEQ ID NO: 38••a base sequence of a heavy chain variable region
“1587”
SEQ ID NOs: 25 to 27••amino acid sequences of light chain CDRs 1 to 3
SEQ ID NOs: 28 to 30••amino acid sequences of heavy chain CDRs 1 to 3
SEQ ID NO: 31••an amino acid sequence of a light chain variable region
SEQ ID NO: 32••an amino acid sequence of a heavy chain variable region
SEQ ID NO: 39••a base sequence of a light chain variable region
SEQ ID NO: 40••a base sequence of a heavy chain variable region

### Example 2

### Analysis of Anti-Serotype G LPS Antibody

### (1) Purification of LPS

Each P. aeruginosa strain of various serotypes shown in Table 3 was suspended in 5 ml of a LB medium. Using this bacterial cell suspension, 1- to 10⁴-fold diluted liquids were prepared by 10-fold serial dilution. These diluted liquids were shaken at 37° C. for 6 hours, for culturing. After the culturing, a bacterial liquid was taken from a diluted liquid which had the largest dilution factor among diluted liquids in which bacterial growth was observed. This bacterial liquid was suspended in a separately prepared LB medium with a dilution factor of 1000, and then shaken at 37° C. overnight for culturing. After the culturing, the liquid was subjected to centrifugation at 5000×g for 20 minutes, and thereby bacterial cells were collected. The weight of the bacterial cells was measured, and then purified water was added to the bacterial cells at 120 mg/ml, in terms of wet weight. Moreover, an equal amount of a 90% solution of phenol (NACALAI TESQUE, INC.) warmed to 68° C. beforehand was added to the bacterial cells, and the mixture was stirred for 20 minutes. Thereafter, the mixture was heated in a water bath at 68° C. for 20 minutes with occasional stirring. Then, after cooling, the mixture was subjected to centrifugation at 5000×g for 20 minutes. The aqueous layer was collected, dialyzed against purified water, and lyophilized. The resulting product was used as each LPS.

### (2) A-Band LPS Purification

LPS G extracted in the above (1) from a P. aeruginosa strain ATCC 27584 of serotype G was used as a raw material. This LPS was again suspended in water for injection, and ultracentrifugation (40000 rpm, 3 hr) was repeated twice to remove nucleic acid. The collected precipitates were lyophilized. The LPS G obtained here was passed through a gel filtration column (HiPrep 26/60 Sephacryl S-200 HR, GE healthcare bioscience, 17-1195-01) for coarse fractionation. For the purification operation, AKTA explore 10S (GE healthcare bioscience) was used. As the mobile phase, a 20 mM Tris-HCl buffer (NACALAI TESQUE, INC., 35406-75) (pH: 8.3) containing 0.2% sodium deoxycholate (NACALAI TESQUE, INC., 10712-54), 0.2 M NaCl (NACALAI TESQUE, INC., 31319-45) and 5 mM EDTA (NACALAI TESQUE, INC., 15105-35) was used. For detection, a differential refractometer (SHIMAZU, RID-10A) was used. The obtained roughly purified fraction was dialyzed against purified water overnight, and then lyophilized. The lyophilized material was again suspended in a 0.5 M NaCl solution, and a 10-fold amount of ethanol was added thereto to thereby cause LPS to be precipitated. The precipitates were again washed with 70% ethanol, to remove the remaining surfactant. Thereafter, the LPS was lyophilized, suspended in a solution of 0.1 N NaOH (NACALAI TESQUE, INC., 31511-05) and 0.2 MNaBH4 (NACALAI TESQUE, INC., 31228-22), and reacted at 37° C. for 24 hr. Thereby, only B-band LPS contained was decomposed according to the method described in Eur. J. BioChem. 167, 203-209 (1987). This reaction liquid was neutralized with a 1% acetic acid (NACALAI TESQUE, INC., 00211-95), concentrated by ultrafiltration (Amicon Ultra-15, MWCO 10000, Millipore), and then subjected again to a gel filtration column (Superdex peptide 10/300 GL, GE healthcare bioscience, 17-5176-01). Fractions eluted using PBS(−) (Sigma-Aldrich Corporation, D1408) as the mobile phase were collected. Thereafter, buffer replacement with purified water and concentration were performed by ultrafiltration. Then, lyophilization was performed to obtain purified A-band LPS.

### (3) Western Blotting and Whole Cell ELISA

### —Western Blotting—

Each of the LPSs obtained from the ATCC strains of various serotypes prepared in Example 2 (1) and the A-band LPS purified in Example 2(2), which were lyophilized, was dissolved in PBS so as to be 1 mg/ml. The solution was mixed with an equal amount of a sample buffer (62.5 mM Tris-HCL (pH: 6.8), 5% 2-mercaptoethanol, 2% SDS, 20% glycerol, 0.005% bromophenol blue), and heated at 100° C. for 10 minutes before use. 10 μl of a LPS was added in each well of 16 well-type 15% SDS-PAGE (XV PANTERA Gel, DRC), and then electrophoresed for 15 minutes. After transfer to a nitrocellulose membrane using a dry gel blotting apparatus (iBlotdry gel blotting system, Invitrogen), blocking was performed at room temperature for 30 minutes using Immunoblock™ (Dainippon Sumitomo Pharma Co., Ltd.). The antibody sample was diluted to 3 μg/ml with 5% Immunoblock™ in TBST (Tris-Buffered Saline containing 0.05% Tween 20), and reacted with the transfer membrane at 4° C. for a day and a night. After washed with TBST for 10 minutes three times, the transfer membrane was immersed in a reaction liquid obtained by diluting a goat anti-human IgG (Fc) antibody HRP conjugate (Kirkegaard & Perry Laboratories, Inc.) with 5% Immunoblock™ in TBST (1:5000), and reaction was performed at 37° C. for 1 hour. Then, after the transfer membrane was washed with TBST for 10 minutes three times, reaction was performed at room temperature for 2 minutes according to the manual of ECL plus Western Blotting Detection System (GE Healthcare, Code: RPN2132). Chemiluminescence was detected by a FLA-3000 fluorescent image analyzer (FUJIFILM Corporation).

Table 3 shows the results. On the membrane to which the antibody 1584 was added as the primary antibody, multiple bands presumably corresponding to B-band LPSs including 0 antigens were observed only from the low molecular weight region to the high molecular weight region of the LPS of clinically frequently encountered serotype G, out of the LPSs obtained from the ATCC strains of 11 serotypes. When LPS obtained from another serotype G strain ATCC 33354 was used, the same results were obtained. Meanwhile, the antibody 1584 did not show any reactivity to the purified A-band LPS. Accordingly, it was confirmed that the antibody 1584 specifically recognized B-band LPS of serotype G LPSs.

TABLE 3

| ATCC | Serotype | 1584 |
|---|---|---|
| 27577 | A/O3 | ND |
| 27578 | B/O2 | ND |
| BAA-47 | B/O5 | ND |
| 27317 | C/O8 | ND |
| 27580 | D/O9 | ND |
| 29260 | E/O11 | ND |
| 27582 | F/O4 | ND |
| 27584 | G/O6 | B band |
| 33354 | G/O6 | B band |
| 27316 | H/O10 | ND |
| 27586 | I/O1 | ND |
| 21636 | M | ND |

ND: not detected

### —Whole Cell ELISA (1)—

Bacterial suspensions used for immobilization were original bacterial suspensions which were prepared by washing, with PBS, bacterial suspensions of P. aeruginosa strains of various serotypes cultured overnight in LB media, and resuspending, in the same buffer, the washed materials so that the absorbance at 595 nm of each 10-fold diluted bacterial suspensions was 0.20 to 0.23. The bacterial suspensions were placed at 100 μl per well of a 96 well ELISA plate (F96 MaxiSorp Nunc-Immuno Plate, Nalge Nunc International K. K.), and immobilization was performed at 4° C. overnight. Thereafter, washing was performed once with 200 μl of TBS. A blocking buffer (TBS containing 2% bovine serum albumin) was added to each of the wells, and blocking was performed for 30 minutes at room temperature. Then, 100 μl of an anti-serotype G LPS antibodies 1572, 1573 and 1587 diluted (5 μg/ml) with a sample buffer (TBS containing 1% bovine serum albumin) was added to each of the wells, and reaction was performed at 37° C. for 2 hours. Thereafter, washing was performed three times each time with 200 μl of a washing buffer (TBS containing 0.05% Tween 20). 100 μl of a secondary antibody, goat anti-human IgG (Fc) antibody HRP conjugate (Kirkegaard & Perry Laboratories, Inc.), diluted 10000-fold with the sample buffer was added to each of the wells, and reaction was performed at 37° C. for 1 hour. Thereafter, washing was performed three times with the washing buffer. 100 μl of a chromogenic substrate (TMB Microwell Peroxidase substrate System, Kirkegaard & Perry Laboratories, Inc.) was added to each of the wells, and reaction was performed in a dark place. Then, the enzymatic reaction was stopped with a 1 M solution of phosphoric acid, and the absorbance at 450 nm was measured. Table 4 shows the results. It was confirmed that, when an absorbance greater than 0.25 was judged as positive, each of the antibodies 1572, 1573, 1584 and 1587 specifically bound to a serotype G strain

TABLE 4

| ATCC | Serotype | 1572 | 1573 | 1584 | 1587 |
|---|---|---|---|---|---|
| 27577 | A/O3 | 0.100 | 0.090 | 0.001 | −0.004 |
| 27578 | B/O2 | −0.040 | −0.022 | 0.005 | −0.004 |
| BAA-47 | B/O5 | 0.049 | 0.032 | −0.001 | −0.005 |
| 33353 | C/O7 | −0.104 | −0.094 | 0.008 | −0.002 |
| 27580 | D/O9 | 0.047 | 0.021 | 0.006 | −0.001 |
| 29260 | E/O11 | −0.126 | −0.094 | 0.016 | 0.079 |
| 27582 | F/O4 | −0.061 | −0.076 | 0.007 | 0.001 |
| 27584 | G/O6 | 1.327 | 1.309 | 1.272 | 1.186 |
| 27316 | H/O10 | −0.132 | −0.114 | 0.018 | −0.002 |
| 27586 | I/O1 | −0.142 | −0.183 | 0.003 | 0.012 |
| 21636 | M | 0.212 | 0.213 | 0.000 | 0.001 |

### —Whole Cell ELISA (2)—

Whole cell ELISA was performed on the antibody 1584 (1.0 μg/ml), using 31 strains in total, which additionally included various serotype strains. Table 5 shows the results. The criteria were as follows: a case with an absorbance of less than 0.25 was marked with −; a case with an absorbance which was 0.25 or more but less than 0.5 was marked with +; a case with an absorbance which was 0.5 or more but less than 0.75 was marked with ++; and a case with an absorbance of 0.75 or more was marked with +++. In such a case, a human immunoglobulin preparation, Venilon (TEIJIN PHARMA LIMITED), which was a control, exhibited no binding capability to the 31 strains. In contrast, the antibody 1584 had +++ only for serotype G strains, and had − for all the strains of the other serotypes, exhibiting a specificity to serotype G strains.

TABLE 5

| ATCC | Serotype | 1584 | | Venilon | |
|---|---|---|---|---|---|
| 27577 | A/O3 | 0.002 | − | 0.025 | − |
| 33350 | A/O3 | 0.006 | − | 0.008 | − |
| 27578 | B/O2 | 0.008 | − | 0.032 | − |
| 33349 | B/O2 | 0.000 | − | 0.068 | − |
| BAA-47 | B/O5 | −0.003 | − | 0.032 | − |
| 33352 | B/O5 | 0.000 | − | 0.045 | − |
| 33363 | B/O16 | 0.005 | − | 0.043 | − |
| 43732 | B/O20 | −0.004 | − | 0.155 | − |
| 33353 | C/O7 | 0.009 | − | 0.003 | − |
| 27317 | C/O8 | 0.007 | − | 0.029 | − |
| 33355 | C/O8 | 0.006 | − | 0.015 | − |
| 27580 | D/O9 | 0.003 | − | 0.020 | − |
| 33356 | D/O9 | 0.004 | − | 0.013 | − |
| 29260 | E/O11 | 0.004 | − | 0.028 | − |
| 33358 | E/O11 | 0.026 | − | 0.031 | − |
| 27582 | F/O4 | −0.002 | − | 0.007 | − |
| 33351 | F/O4 | 0.006 | − | 0.018 | − |
| 27584 | G/O6 | 1.204 | +++ | 0.037 | − |
| 33354 | G/O6 | 1.224 | +++ | 0.026 | − |
| 27316 | H/O10 | −0.008 | − | 0.008 | − |
| 33357 | H/O10 | 0.001 | − | 0.014 | − |
| 27586 | I/O1 | 0.001 | − | 0.012 | − |
| 33348 | I/O1 | 0.008 | − | 0.009 | − |
| 33362 | J/O15 | −0.001 | − | 0.016 | − |
| 33360 | K/O13 | 0.016 | − | 0.012 | − |
| 33361 | K/O14 | 0.004 | − | 0.024 | − |
| 33359 | L/O12 | 0.006 | − | 0.023 | − |
| 21636 | M | 0.006 | − | 0.028 | − |
| 33364 | N/O17 | 0.027 | − | 0.020 | − |
| 43390 | O18 | 0.003 | − | 0.014 | − |
| 43731 | O19 | 0.005 | − | 0.009 | − |

### (4) Cross-Reaction Test

To test cross-reaction of the anti-serotype G LPS antibody 1584 (1.0 μg/ml), whole cell ELISA was performed using various Gram-negative and Gram-positive pathogenic bacteria in the same method as in the above (1). Table 6 shows the results. The anti-serotype G LPS antibody 1584 specifically recognized and bound strongly to the serotype G/O6 ATCC 27584 strain, but did not react with other bacterial strains.

TABLE 6

| | 1584 | Venilon | Synagis |
|---|---|---|---|
| P. aeruginosa ATCC 27577 (A/O3) | 0.013 | 0.020 | 0.006 |
| P. aeruginosa ATCC BAA-47 (B/O5) | 0.005 | 0.016 | 0.008 |
| P. aeruginosa ATCC 29260 (E/O11) | 0.008 | 0.014 | 0.004 |
| P. aeruginosa ATCC 27584 (G/O6) | 0.601 | 0.013 | 0.002 |
| P. aeruginosa ATCC 27586 (I/O1) | 0.010 | 0.015 | 0.003 |
| P. aeruginosa ATCC 21636 (M) | 0.004 | 0.016 | 0.001 |
| P. alcaligenes ATCC 14909 | 0.043 | 0.021 | 0.006 |
| P. aureofaciens ATCC 13985 | 0.019 | 0.017 | 0.005 |
| P. chlororaphis ATCC 9446 | 0.021 | 0.007 | 0.003 |
| Acinetobacter baumannii ATCC BAA-1710 | −0.005 | 0.012 | −0.005 |
| Stenotrophomonas maltophilia ATCC | 0.085 | 0.021 | −0.002 |
| 13637 | | | |
| Burkholderia cepacia ATCC 25416 | 0.017 | 0.011 | −0.001 |
| Bacillus subtillis ATCC 6633 | 0.011 | 0.047 | −0.002 |
| Escherichia coli ATCC 25922 | 0.015 | 0.025 | 0.001 |
| Klebsiella pneumoniae ATCC 700603 | 0.003 | 0.018 | −0.004 |

### (5) Agglutination Activity

Using the P. aeruginosa ATCC 27584 strain (serotype G/O6), the agglutination activity of the antibody 1584 was measured. This strain was cultured on a trypticase soy agar medium at 37° C. overnight. Then, after several colonies were suspended in a LB medium, the medium was shaken at 37° C. overnight for culturing. The bacterial culture was washed with PBS and resuspended in PBS. Then, a phosphate buffer containing 4% paraformaldehyde (Wako Pure Chemical Industries, Ltd.) was added thereto, and inactivation treatment was performed for 30 minutes or more. This treated product was used for the test. The inactivated ATCC 27584 strain was suspended in PBS so as to be 2 mg/ml of protein concentration. The antibody 1584 (concentration of IgG in the original liquid: 3.09 mg/ml) was serially diluted with PBS. Equal amounts (8 μl) of the inactivated ATCC 27584 strain suspension and the serially diluted antibody 1584 were mixed with each other on a 96-well round bottom plate. Each mixture was stood at 37° C. for 1 hour or more, or at room temperature overnight or longer. Then, agglutination of bacterial cells was judged.

As a result, the agglutination titer of the antibody 1584 was 64, in other words, agglutination was observed up to 64-fold dilution, and the agglutination titer per amount (μg) of IgG was 166. Meanwhile, the agglutination titer of an immunoglobulin preparation, Venilon, (50 mg/ml, TEIJIN PHARMA LIMITED), which was a control, was 32, in other words, agglutination was observed up to 32-fold dilution, and the agglutination titer per amount (μg) of IgG was 2.56.

### (6) Opsonic Activity

### —Test 1—

The serotype G P. aeruginosa strain ATCC 33354 was cultured in a LB medium overnight. The bacterial culture was fixed with 4% paraformaldehyde, and suspended in a 1 mM solution of fluorescein-4-isothiocyanate (FITC) at room temperature for 1 hour to perform labeling. By a density gradient centrifugation method using a Mono-Poly resolving medium (DS Pharma Biomedical Co. Ltd.), human polymorphonuclear leukocytes (hereinafter, referred to as PMN) were purified from 50 ml of blood collected using citric acid from healthy donors, and were prepared to have a concentration of 5×10⁶ cells/ml. 20 μl of the anti-serotype G LPS antibody 1584 and the FITC-labeled P. aeruginosa strain (30 μl, 5×10⁶) were added in a 96-well round-bottom plate, and incubated at 37° C. for 15 minutes. Thereafter, as complements, baby rabbit serum (10 μl) and the PMN (40 μl, 2×10⁵ cells) were added, and the mixture was further incubated for 30 minutes to carry out phagocytosis. The plate was transferred onto ice, and thereby the reaction was stopped. The fluorescence of bacteria attaching to the cell surfaces was quenched by PBS containing 0.2% trypan blue (100 μl), and then the cells were fixed with 0.5% paraformaldehyde. Using a flow cytometer (BECKMAN COULTER), the fluorescence (mean fluorescence intensity, hereinafter abbreviated as MFI) of the cells was measured. The opsonic activity was calculated as a value obtained by subtracting the fluorescence intensity due to the intrinsic fluorescence of the PMN from the fluorescence intensity of PMN which incorporated the FITC-labeled P. aeruginosa strain.

As a result, for the serotype G strain ATCC 33354, the MFI value of a group to which no antibody was added was 18.15, and the MFI value of a group to which the anti-serotype G LPS antibody 1584 was added increased concentration-dependently, where the MFI value was 367.55 at 30 μg/ml, and the EC50 was 0.10 μg/ml. The MFI value of an immunoglobulin preparation, Venilon (TEIJIN PHARMA LIMITED), which was used as a control, was 277.45 at 1000 μg/ml.

The above-described results showed that the anti-serotype G LPS antibody 1584 had a strong opsonic activity against a strain of serotype G, which is clinically frequently encountered.

### —Test 2—

The serotype G P. aeruginosa strain ATCC 27584 was cultured on a Mueller-Hinton agar medium overnight. Then, 3 colonies were picked up therefrom, inoculated in a Luria-Bertani culture medium, and cultured at 37° C. for 16 hours with shaking (180 rpm). The culture medium was subjected to centrifugation (2,000×g, 10 minutes, at room temperature). The resultant material was washed once with phosphate-buffered saline (PBS), and then suspended in a 1 mM solution of fluorescein-4-isothiocyanate (FITC) at room temperature for 1 hour to perform labeling. By a density gradient centrifugation method using a Mono-Poly resolving medium (DS Pharma Biomedical Co. Ltd.), human polymorphonuclear leukocytes (hereinafter, referred to as PMN) were purified from 50 ml of blood collected using citric acid from healthy donors, and were prepared to have a concentration of 5×10⁶ cells/ml. 20 μl of each of the anti-serotype G LPS antibodies 1573, 1572 and 1587 with the FITC-labeled P. aeruginosa strain (30 μl, 5×10⁶) was added in a 96-well round-bottom plate, and incubated at 37° C. for 15 minutes. Thereafter, as complements, baby rabbit serum (10 μl) and the PMN (40 μl, 2×10⁵ cells) were added, and the mixture was further incubated for 30 minutes to carry out phagocytosis. The plate was transferred onto ice, and thereby the reaction was stopped. The fluorescence of bacteria attaching to the cell surfaces was quenched by PBS containing 0.2% trypan blue (100 μl), and then the cells were fixed with 0.5% paraformaldehyde. Using a flow cytometer (BECKMAN COULTER), the fluorescence (mean fluorescence intensity, hereinafter abbreviated as MFI) of the cells was measured. The opsonic activity was calculated as a value obtained by subtracting the fluorescence intensity due to the intrinsic fluorescence of the PMN from the fluorescence intensity of PMN which incorporated the FITC-labeled P. aeruginosa strain.

As a result, the experiments using the antibody 1573 showed that, for the serotype G P. aeruginosa strain ATCC 27584, the MFI value of a group to which no antibody was added was 8.54, and the MFI value of a group to which the antibody 1573 was added increased concentration-dependently, where the MFI value was 37.94 at 30 μg/ml, and the EC50 was 0.72 μg/ml. The MFI value of an immunoglobulin preparation, Venilon (TEIJIN PHARMA LIMITED), which was used as a control, was 15.14 at 1000 μg/ml.

Meanwhile, the experiments using the antibody 1572 and the antibody 1587 gave the following results. For the serotype G P. aeruginosa strain ATCC 27584, the MFI value of a group to which no antibody was added was 20.18, and the MFI value of a group to which the antibody 1572 was added increased concentration-dependently, where the MFI value was 107.68 at 30 μg/ml, and the EC50 was 1.30 μg/ml. Also for the serotype G P. aeruginosa strain ATCC 27584, the MFI value of a group to which the antibody 1587 was added increased concentration-dependently, where the MFI value was 74.13 at 30 μg/ml, and the EC50 was 2.59 μg/ml.

The above-described results showed that the anti-serotype G LPS antibodies 1573, 1572 and 1587 had opsonic activities against a P. aeruginosa strain of serotype G.

### (7) Effect on Systemic Infection Model

Neutropenic mice were prepared as follows.

Cyclophosphamide (Sigma-Aldrich) was intraperitoneally injected into each 6-week-old BALB/c male mouse (Charles river laboratories Japan, inc., n=12) at 125 mg/kg three times in total on days −5, −2 and 0, where the day of infection was designated as day 0. Thereby, neutrophils in the peripheral blood were decreased. Into the mouse, the ATCC 27584 strain (serotype G/O6) was inoculated intraperitoneally at 1.575×10⁴ to 2.15×10⁴ cfu/mouse (approximately 50 to 70 LD50), to thereby induce a systemic infection. Immediately thereafter, a sample was administered via tail vein at 200 μl/mouse, and a protective activity against the infection was judged on the basis of the survival thereof 7 days after the inoculation. As a result, the survival rates, on day 7 after the infection, of control groups to which an immunoglobulin preparation, Venilon (TEIJIN PHARMA LIMITED), was administered at 5, 50, 500 and 2500 μg/mouse were 0, 8.3, 25 and 66.7%, respectively, and the ED50 was estimated to be 1288.88 μg/mouse. In contrast, the survival rates, on day 7 after the infection, of groups to which the anti-serotype G LPS antibody 1572 was administered at 0.5, 1, 2, 5, 20 and 50 μg/mouse were 16.7, 8.3, 16.7, 41.7, 75 and 58.3%, respectively, showing a strong protective activity against the infection, and the ED50 was estimated to be 12.24 μg/mouse. The survival rates, on day 7 after the infection, of groups to which the anti-serotype G LPS antibody 1573 was administered at 0.5, 1, 2, 5, 20 and 50 μg/mouse were 0, 0, 66.7, 41.7, 75 and 75%, respectively, showing a strong protective activity against the infection, and the ED50 was estimated to be 6.57 μg/mouse. Meanwhile, the survival rates, on day 7 after the infection, of groups to which the anti-serotype G LPS antibody 1584 was administered at 0.5, 1, 2, 5, 20 and 50 μg/mouse were 25, 33.3, 41.7, 41.7, 75 and 83.3%, respectively, showing a strong protective activity against the infection, and the ED50 was estimated to be 4.01 μg/mouse.

### (8) Effect on Systemic Infection Model 2

Neutropenic mice were prepared as follows. Cyclophosphamide (hereinafter referred to as CY, Sigma-Aldrich) was intraperitoneally injected into each 6-week-old BALB/c male mouse (Charles River Laboratories Japan, Inc., n=12) at 125 mg/kg three times in total on days −5, −2 and 0, where the day of infection was designated as day 0. Thereby, neutrophils in the peripheral blood were decreased. Into the mouse, the ATCC 27584 strain (serotype G/O6) suspended in 250 of saline was inoculated intraperitoneally at 1.75×10⁴ cfu/mouse (approximately 55 LD50), to thereby induce a systemic infection. Immediately after the inoculation, a sample was administered via tail vein at 200 μl/mouse, and a protective activity against the infection was evaluated on the basis of the survival thereof 7 days after the inoculation. As a result, the survival rates, on day 7 after the infection, of control groups to which an immunoglobulin preparation, Venilon (TEIJIN PHARMA LIMITED), was administered at 200, 1000, 5000, 10000, and 25000 μg/mouse were 0, 0, 50, 0, and 66.7%, respectively, and the ED50 was estimated to be 18579.04 μg/mouse. The survival rates, on day 7 after the infection, of groups to which an anti-PcrV antibody M166 was administered at 1.6, 8, and 40 μg/mouse were all 0%, and the ED50 was estimated to be >40 μg/mouse. In contrast, the survival rates, on day 7 after the infection, of groups to which the antibody 1584 was administered at 0.064, 0.32, 1.6, 8, and 40 μg/mouse were 0, 50, 50, 100, and 50%, respectively, showing a strong protective activity against the infection, and the ED50 was estimated to be 1.73 μg/mouse.

### INDUSTRIAL APPLICABILITY

An antibody of the present invention has an excellent antibacterial activity against P. aeruginosa, and hence can be used for treatment or prevention of P. aeruginosa infections. The antibody of the present invention is a human antibody, and hence is highly safe. Accordingly, the antibody of the present invention is extremely useful for medical care. Furthermore, the monoclonal antibody of the present invention can be applied for diagnosis of P. aeruginosa infections, detection or screening of P. aeruginosa strains of various serotypes, and the like.

1. An antibody which recognizes B-band LPS of lipopolysaccharides of P. aeruginosa, and which substantially binds to a surface of a P. aeruginosa strain of serotype G, but does not substantially binds to any one of surfaces of P. aeruginosa strains of serotype A, B, C, D, E, F, H, I, and M.

2. The antibody according to claim 1, which has an opsonic activity against a P. aeruginosa strain of serotype G.

3. The antibody according to claim 2, wherein an EC50 of an opsonic activity against a P. aeruginosa strain identified by ATCC 33354 is 0.5 μg/ml or less.

4. The antibody according to claim 2, wherein an EC50 of an opsonic activity against a P. aeruginosa strain identified by ATCC 27584 is 3 μg/ml or less.

5. The antibody according to claim 1, which has an agglutination activity against a P. aeruginosa strain of serotype G.

6. The antibody according to claim 5, wherein an agglutination titer per amount (μg) of IgG against a P. aeruginosa strain identified by ATCC 27584 is 100 or more.

7. The antibody according to claim 1, which has an antibacterial effect against a systemic infection with a P. aeruginosa strain of serotype G.

8. The antibody according to claim 7, wherein an ED50 of an antibacterial effect on a neutropenic mouse model of systemic infection with a P. aeruginosa strain identified by ATCC 27584 is not more than 1/100 of that of Venilon.

9. The antibody which has any one of the following features (a) to (d):
(a) comprising
a light chain variable region including amino acid sequences described in SEQ ID NOs: 1 to 3 or the amino acid sequences described in SEQ ID NOs: 1 to 3 in at least one of which one or more amino acids are substituted, deleted, added, and/or inserted, and
a heavy chain variable region including amino acid sequences described in SEQ ID NOs: 4 to 6 or the amino acid sequences described in SEQ ID NOs: 4 to 6 in at least one of which one or more amino acids are substituted, deleted, added, and/or inserted;
(b) comprising
a light chain variable region including amino acid sequences described in SEQ ID NOs: 9 to 11 or the amino acid sequences described in SEQ ID NOs: 9 to 11 in at least one of which one or more amino acids are substituted, deleted, added, and/or inserted, and
a heavy chain variable region including amino acid sequences described in SEQ ID NOs: 12 to 14 or the amino acid sequences described in SEQ ID NOs: 12 to 14 in at least one of which one or more amino acids are substituted, deleted, added, and/or inserted;
(c) comprising
a light chain variable region including amino acid sequences described in SEQ ID NOs: 17 to 19 or the amino acid sequences described in SEQ ID NOs: 17 to 19 in at least one of which one or more amino acids are substituted, deleted, added, and/or inserted, and
a heavy chain variable region including amino acid sequences described in SEQ ID NOs: 20 to 22 or the amino acid sequences described in SEQ ID NOs: 20 to 22 in at least one of which one or more amino acids are substituted, deleted, added, and/or inserted; and
(d) comprising
a light chain variable region including amino acid sequences described in SEQ ID NOs: 25 to 27 or the amino acid sequences described in SEQ ID NOs: 25 to 27 in at least one of which one or more amino acids are substituted, deleted, added, and/or inserted, and
a heavy chain variable region including amino acid sequences described in SEQ ID NOs: 28 to 30 or the amino acid sequences described in SEQ ID NOs: 28 to 30 in at least one of which one or more amino acids are substituted, deleted, added, and/or inserted.

10. The antibody which has any one of the following features (a) to (d):
(a) comprising
a light chain variable region including an amino acid sequence described in SEQ ID NO: 7 or the amino acid sequence described in SEQ ID NO: 7 in which one or more amino acids are substituted, deleted, added, and/or inserted, and
a heavy chain variable region including an amino acid sequence described in SEQ ID NO: 8 or the amino acid sequences described in SEQ ID NO: 8 in which one or more amino acids are substituted, deleted, added, and/or inserted;
(b) comprising
a light chain variable region including an amino acid sequence described in SEQ ID NO: 15 or the amino acid sequences described in SEQ ID NO: 15 in which one or more amino acids are substituted, deleted, added, and/or inserted, and
a heavy chain variable region including an amino acid sequence described in SEQ ID NO: 16 or the amino acid sequences described in SEQ ID NO: 16 in which one or more amino acids are substituted, deleted, added, and/or inserted;
(c) comprising
a light chain variable region including an amino acid sequence described in SEQ ID NO: 23 or the amino acid sequences described in SEQ ID NO: 23 in which one or more amino acids are substituted, deleted, added, and/or inserted, and
a heavy chain variable region including an amino acid sequence described in SEQ ID NO: 24 or the amino acid sequences described in SEQ ID NO: 24 in which one or more amino acids are substituted, deleted, added, and/or inserted; and
(d) comprising
a light chain variable region including an amino acid sequence described in SEQ ID NO: 31 or the amino acid sequences described in SEQ ID NO: 31 in which one or more amino acids are substituted, deleted, added, and/or inserted, and
a heavy chain variable region including an amino acid sequence described in SEQ ID NO: 32 or the amino acid sequence described in SEQ ID NO: 32 in which one or more amino acids are substituted, deleted, added, and/or inserted.

11. A peptide comprising a light chain or a light chain variable region of the antibody which has any one of the following features (a) to (d):
(a) comprising amino acid sequences described in SEQ ID NOs: 1 to 3 or the amino acid sequences described in SEQ ID NOs: 1 to 3 in at least one of which one or more amino acids are substituted, deleted, added, and/or inserted;
(b) comprising amino acid sequences described in SEQ ID NOs: 9 to 11 or the amino acid sequences described in SEQ ID NOs: 9 to 11 in at least one of which one or more amino acids are substituted, deleted, added, and/or inserted;
(c) comprising amino acid sequences described in SEQ ID NOs: 17 to 19 or the amino acid sequences described in SEQ ID NOs: 17 to 19 in at least one of which one or more amino acids are substituted, deleted, added, and/or inserted; and
(d) comprising amino acid sequences described in SEQ ID NOs: 25 to 27 or the amino acid sequences described in SEQ ID NOs: 25 to 27 in at least one of which one or more amino acids are substituted, deleted, added, and/or inserted.

12. A peptide comprising a light chain or a light chain variable region of the antibody, the peptide having any one of the following features (a) to (d):
(a) comprising an amino acid sequence described in SEQ ID NO: 7 or the amino acid sequence described in SEQ ID NO: 7 in which one or more amino acids are substituted, deleted, added, and/or inserted;
(b) comprising an amino acid sequence described in SEQ ID NO: 15 or the amino acid sequences described in SEQ ID NO: 15 in which one or more amino acids are substituted, deleted, added, and/or inserted;
(c) comprising an amino acid sequence described in SEQ ID NO: 23 or the amino acid sequences described in SEQ ID NO: 23 in which one or more amino acids are substituted, deleted, added, and/or inserted; and
(d) comprising an amino acid sequence described in SEQ ID NO: 31 or the amino acid sequences described in SEQ ID NO: 31 in which one or more amino acids are substituted, deleted, added, and/or inserted.

13. A peptide comprising a heavy chain or a heavy chain variable region of the antibody, which has any one of the following features (a) to (d):
(a) comprising amino acid sequences described in SEQ ID NOs: 4 to 6 or the amino acid sequences described in SEQ ID NOs: 4 to 6 in at least one of which one or more amino acids are substituted, deleted, added, and/or inserted;
(b) comprising amino acid sequences described in SEQ ID NOs: 12 to 14 or the amino acid sequences described in SEQ ID NOs: 12 to 14 in at least one of which one or more amino acids are substituted, deleted, added, and/or inserted;
(c) comprising amino acid sequences described in SEQ ID NOs: 20 to 22 or the amino acid sequences described in SEQ ID NOs: 20 to 22 in at least one of which one or more amino acids are substituted, deleted, added, and/or inserted; and
(d) comprising amino acid sequences described in SEQ ID NOs: 28 to 30 or the amino acid sequences described in SEQ ID NOs: 28 to 30 in at least one of which one or more amino acids are substituted, deleted, added, and/or inserted.

14. A peptide comprising a heavy chain or a heavy chain variable region of the antibody, which has any one of the following features (a) to (d):
(a) comprising an amino acid sequence described in SEQ ID NO: 8 or the amino acid sequences described in SEQ ID NO: 8 in which one or more amino acids are substituted, deleted, added, and/or inserted;
(b) comprising an amino acid sequence described in SEQ ID NO: 16 or the amino acid sequences described in SEQ ID NO: 16 in which one or more amino acids are substituted, deleted, added, and/or inserted;
(c) comprising an amino acid sequence described in SEQ ID NO: 24 or the amino acid sequences described in SEQ ID NO: 24 in which one or more amino acids are substituted, deleted, added, and/or inserted; and
(d) comprising an amino acid sequence described in SEQ ID NO: 32 or the amino acid sequence described in SEQ ID NO: 32 in which one or more amino acids are substituted, deleted, added, and/or inserted.

15. An antibody which binds to an epitope, in B-band LPS of lipopolysaccharides of a P. aeruginosa strain of serotype G, of an antibody described in any one of the following (a) to (d):
(a) an antibody comprising a light chain variable region including an amino acid sequence described in SEQ ID NO: 7 and a heavy chain variable region including an amino acid sequence described in SEQ ID NO: 8;
(b) an antibody comprising a light chain variable region including an amino acid sequence described in SEQ ID NO: 15 and a heavy chain variable region including an amino acid sequence described in SEQ ID NO: 16;
(c) an antibody comprising a light chain variable region including an amino acid sequence described in SEQ ID NO: 23 and a heavy chain variable region including an amino acid sequence described in SEQ ID NO: 24; and
(d) an antibody comprising a light chain variable region including an amino acid sequence described in SEQ ID NO: 31 and a heavy chain variable region including an amino acid sequence described in SEQ ID NO: 32.

16. A DNA which codes the antibody or the peptide according to claim 1.

17. A hybridoma which produces the antibody according to claim 1, and 15.

18. A pharmaceutical composition for a disease associated with P. aeruginosa, the pharmaceutical composition comprising:
the antibody according to claim 1; and optionally
at least one pharmaceutically acceptable carrier and/or diluent.

19. The pharmaceutical composition according to claim 18, wherein the disease associated with P. aeruginosa is a systemic infectious disease caused by a P. aeruginosa infection.

20. The pharmaceutical composition according to claim 18, wherein the disease associated with P. aeruginosa is a pulmonary infectious disease caused by a P. aeruginosa infection.

21. A diagnostic agent for detection of P. aeruginosa, the diagnostic agent comprising: the antibody according to claim 1.

22. A kit for detection of P. aeruginosa, the kit comprising: the antibody according to claim 1.
